(19) United States
(12) Patent Application Publication
(10) Pub. No.: US 2016/0340658 A1
(43) Pub. Date: Nov. 24, 2016

(54) PTERIS VITTATA PHYTASE NUCLEOTIDE AND AMINO ACID SEQUENCES AND METHODS OF USE

(71) Applicant: UNIVERSITY OF FLORIDA RESEARCH FOUNDATION, INC., Gainesville, FL (US)

(72) Inventors: JASON LESSL, GAINESVILLE, FL (US); LENA Q. MA, GAINESVILLE, FL (US); BALASUBRAMANI RATHINASABAPATHIR, GAINESVILLE, FL (US); CHARLES L. GUY, GAINESVILLE, FL (US)

(21) Appl. No.: 15/116,534

(22) PCT Filed: Nov. 7, 2014
(86) PCT No.: PCT/US2014/064586

§ 371 Date: Aug. 4, 2016

Related U.S. Application Data
(60) Provisional application No. 61/935,387, filed on Feb. 4, 2014.

(51) Int. Cl. C12N 9/16; A23K 20/189; C05F 11/00

(52) U.S. Cl. CPC C12N 9/16; C12Y 301/03008; C05F 11/00; A23K 20/189

(57) ABSTRACT

The present disclosure provides isolated and synthetic DNA and cDNA molecules encoding a phytase from the root of Pteris vittata (PV); root PV phytase proteins and peptides; root PV phytase antisense molecules, vectors, transgenic cells and plants containing root PV phytase nucleic acid molecules, isolated polypeptides, or antisense molecules; genetic markers for root PV phytase; and methods of using these nucleic acid or polypeptide molecules to improve phosphorus utilization from phytate by plants and animals.

### CROSS-REFERENCE TO RELATED APPLICATIONS

This application claims the benefit of U.S. provisional application Ser. No. 61/935,387 filed on Feb. 4, 2014, having the title “Pteris Vittata Phytase and Amino Acid Sequences and Methods of Use,” the disclosure of which is incorporated by reference herein in its entirety.

### SEQUENCE LISTING

This application contains a sequence listing filed in electronic form as an ASCII.txt file entitled 02106537.txt, created on Nov. 6, 2014, and having a size of 68,543 bytes. The content of the sequence listing is incorporated herein in its entirety.

### BACKGROUND

Phosphorus (P) is an essential element for plant, animal, and human growth. Phosphorus supplementation, either in the form of fertilizer or feed additives, has long been recognized as necessary to maintain profitable crop and animal production. Supplementation is necessary because, despite being abundant in the lithosphere, phosphorus is one of the most limiting nutrients affecting agricultural production around the world. See Cordell, D., J. et al. Global Environmental Change. 2009, 19:292-305. Phosphorus is a limiting nutrient for plants and non-ruminants because the majority of phosphorus is unavailable for utilization. Phosphorus exists in two forms: 1) Organic P (Pₒ), and 2) Inorgainc P (Pᵢ). Pₒ accounts for 30-80% of total soil phosphorus, predominately as phytate [myo-inositol 1,2,3,4,5,6-hexakisphosphate]. See Organic phosphorus in the Environment, Eds. B. L. Turner, E. Frossard, and D. Baldwind. CAB International. 2005, pp 165-184. Plants, non-ruminant animals, and humans require inorganic phosphorus because they cannot effectively absorb the abundant phytate. Phytate makes up >25% and 60-86% of the total phosphorus in soil and feed/food, respectively. See Id. and Lei, X. G., et al. Annu Rev. Anim. Biosci. 2013, 1:283-309. Plants have intracellular phosphatases that are involved in utilization of Pᵢ reserves. In plant roots, phytase enzymes can occur in the apoplast but are often localized to the cell wall, epidermal cells, and apical meristem. Despite this, plant root phosphatases are unable to hydrolyze sufficient Pᵢ to maintain growth owing to poor substrate availability in soils due to sorption and precipitation, proteolytic breakdown, and/or limited capacity to effectively exude Pᵢ mobilizing enzymes. Non-ruminant animals and humans lack phytase, which is a phosphatase that removes phosphorus from phytate making phosphorous available for absorption by the intestine.

Poor phosphorus utilization by plants and animals contributes to eutrophication. Eutrophication causes degradation of lakes or streams as a result of nutrient enrichment See A. N. Sharpley, T. et al. “Agriculture Phosphorus and Eutrophication” 2^{nd} Edition, September 2003, published by the United States Department of Agriculture ARS-149. Eutrophication has been identified as the main cause of impaired surface water quality and is accelerated by phosphorus. See Schindler, D. W. Science. 1977, 195:260-262 and Sharpley, A. N., et al. J. Envir. Qual. 1994, 23:437-451. Unutilized phosphorus is excreted in animal waste, which is used as fertilizer. Unutilized phosphorus from animal waste and inorganic fertilizers accumulates on the land. Excess phosphorus leaches into surface and below-ground waterways, which contributes to eutrophication.

For at least the past three decades, the agriculture industry has been working to reduce phosphorus pollution from agriculture. Despite several decades of effort in multiple disciplines phosphorus-mediated eutrophication has yet to be remedied. As such, there exists a need for improved phosphorus utilization strategies to protect the environment while still sustaining agriculture production.

### SUMMARY

Briefly described, embodiments of the present disclosure provide isolated nucleotide and cDNA molecules encoding a phytase from the roots of Pteris vittata (PV), isolated polypeptide molecules corresponding to a phytase from the roots of PV, polypetides capable of cleaving phosphate from phytate at temperatures of about 100° C., antisense molecules capable of inhibiting production of root PV phytase, vectors including the root PV phytase cDNA or antisense molecules, cells and plants including the root PV phytase DNA or antisense molecules, methods of increasing or decreasing the amount of root PV phytase expressed by a plant or cell, and genetic markers for root PV phytase genes.

The present disclosure provides cDNA molecules encoding a root PV phytase capable of cleaving phosphate from phytate, where the cDNA molecules have about 90% or greater sequence identity with SEQ ID NO: 2. In some embodiments, the cDNA is operatively linked to a regulatory sequence. The present disclosure also provides isolated polypeptides having at least about 90% or greater sequence identity to SEQ ID NO: 3. In some embodiments, the isolated polypeptide cleaves phosphate from phytate at a temperature of greater than about 70° C. The present disclosure also provides vectors including a cDNA molecule encoding a root PV phytase having at least about 90% sequence identity to SEQ ID NO: 2.

The present disclosure also provides cells and transgenic plants transformed with vectors including the cDNA molecule encoding root PV phytase with at least about 90% percent sequence identity with SEQ ID NO: 2. In some embodiments, the transformed cells and plants express a root PV phytase polypeptide with at least about 90% sequence identity with SEQ ID NO: 3. In further embodiments, the expressed root PV phytase polypeptide cleaves phosphate from phytate at temperatures of greater than about 70° C. In some embodiments the transformed cells are mixed with a soil. In other embodiments, parts of the transgenic plants expressing root PV phytase are included in an animal feed.

The present disclosure also provides for a purified recombinant root PV phytase having at least about 90% sequence identity to SEQ ID NO: 3. In some embodiments, the purified recombinant root PV phytase cleaves phosphate from phytate. In further embodiments, the purified recombinant root PV phytase cleaves phosphate from phytate at temperatures of about 70° C. or greater. Also provided herein are methods for making a purified recombinant root PV phytase from transformed cells. In some embodiments, the purifiedrecombinant root PV phytase is included in an animal feed.

Other compositions, plants, methods, features, and advantages of the present disclosure will be or become apparent to one with skill in the art upon examination of the following drawings and detailed description. It is intended that all such additional compositions, plants, methods, features, and advantages be included within this description, and be within the scope of the present disclosure.

### BRIEF DESCRIPTION OF THE DRAWINGS

Further aspects of the present disclosure will be readily appreciated upon review of the detailed description of its various embodiments, described below, when taken in conjunction with the accompanying drawings.

FIGS. 1A and 1B show the effect of arsenate on phytase (FIG. 1A) and phosphatase (FIG. 1B) activity in root extracts from P. vittata (PV), P. ensiformis (PE), and purified wheat phytase (WP). Enzyme activities were determined by incubating samples in 5 mM phytate or pNPP suspensions buffered at pH 5.0 with increasing concentrations of arsenate. Specific activity values for PV, PE, and WP were 46.7, 42.7, and 51.8 nmol Pᵢ mg⁻¹ protein min⁻¹ for phytase and 79.5, 149, and 163 nmol pNP mg⁻¹ protein min⁻¹ for phosphatase respectively. Data are the means of ten replicates with bars representing standard error.

FIGS. 2A and 2B show the effect of temperature on root P. vittata phytase (FIG. 2A) or root P. vittata phosphatase (FIG. 2B). Enzyme activities from extracts of P. vittata (PV), P. ensiformis (PE) and purified wheat phytase (WP) were determined by incubating 5 mM phytate or pNPP suspensions buffered at pH 5.0 following 10 min pretreatments in a water bath held at 40, 60, 80, or 100° C. Data are the means of ten replicates with bars representing standard error.

FIG. 3 shows the activities of phosphatase and phytase in the fronds and the rhizomes of P. vittata (PV) and P. ensiformis (PE) following 3 day treatment in phosphate and arsenate.

FIG. 4 shows the activities of phosphatase and phytase in the root tissues of P. vittata (PV) and P. ensiformis (PE) following 3 day treatment in phosphate and arsenate.

FIG. 5 shows the fresh weights of plants (L. sativa, A. schoenoprasum, T. subterraneum, P. ensiformis, T. kunthii, and P. vittata) after 15 d or 40 d of growth on sterile Murashige & Skoog media containing no P (control), arsenate (As), phosphate (Pᵢ), and phytate (P₆).

FIGS. 6A-6D show plant growth at 15 d (L. sativa (FIG. 6B) and T. subterraneum (FIG. 6D)) or 40 d (P. vittata (FIG. 6A) and P. ensiformis FIG. 6C)) on sterile Murashige & Skoog media containing phosphate (P), phytate, or phosphate with arsenate (P+As).

FIG. 7 shows sporophyte tissues produced by P. vittata gametophytes after 40 d of growth on amended media containing 0.6 mM phytate and arsenate.

FIG. 8 shows total concentration of phosphorus (P) and arsenate (As) in P. vittata gametophytes grown on Murashige & Skoog media with 0.6 mM phosphate (Pᵢ), phytate (P₆), and arsenate (As) for 40 d.

FIGS. 9A and 9B show the effect of phytate on phytase activity in P. vittata root exudates (FIG. 9A) and gametophytes (FIG. 9B). Phytase activities were determined from P. vittata root exudates (FIG. 9A) and gametophytes (FIG. 9B) grown with phosphate (Pᵢ), phytate (P₆), and arsenate (As). Data represent the mean of eight replicates with standard error and bars with the same letters are not significantly different.

FIGS. 10A and 10B show phytase activity (FIG. 10A) remaining in soil suspensions after mixing with root enzyme extracts from the roots of P. vittata (PV), P. ensiformis (PE), or purified wheat phytase (WP) for 2 h and the response of PV extracts to soil over a 24 h period (FIG. 13B). Data are the means of five replicates with bars representing standard error.

FIG. 11 shows protein content in exudates from roots of P. vittata treated with phytate (PA), inorganic P (Pi), and/or arsenate (As).

FIG. 12 shows phytase activity in gametophyte root exudates and sporophyte exudates after treatment with inorganic P (Pi), Pi and arsenate (As), or phytate IHP)

FIG. 13 shows activity of root PV phytase (PV), P. ensiformis (PE) phytase, or purified wheat phytase (WP) in different soils demonstrating activity of root PV phytase even when sorbed to soil particles. Activity of the various phytases is represented on the vertical axis. Soil sample or control is represented on the horizontal axis.

FIG. 14 shows activity of root PV phytase at varying pH. Phytase activity is shown on the vertical axis. pH is shown on the horizontal axis.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

### DISCUSSION

Phytases are a class of phosphatases that initiate stepwise removal of phosphate from phytic acid and its salt, phytate. All phytases enable phosphate monoester hydrolysis of phytic acid/phytate. As animal diets became plant based (i.e. soybean based) over the second half of the 20^{th} century, the amount of excreted phosphorus increased because phosphorus in these plant feeds was in the form of phytate. As a result, increasing amounts of Pᵢ were supplemented into non-ruminant diets to meet the phosphorus requirements of the animals. Further, phytate acts as an antinutrient by chelating other micronutrients, such as iron. In this way phytate reduces the availability of these micronutrients, which impacts the growth of plants and animals. Efforts to develop a commercial phytase supplement for animal feeds began as early as the 1960's. See Wodzinski R. J. and A. H. Ullah. Adv. Appl. Microbiol. 1996, 42:263-301. Currently, a majority of swine and poultry diets are supplemented with phytases and the phytase market represents more than 60% of the total feed-enzyme market. See Adeola, O. and A. J. Cowieson. J. Anim. Sci. 2011, 89:3189-3218. The improved performance of animals fed diets supplemented with phytase is attributed to both improved phosphorus and other micronutrient utilization.

The substantial loss of phytase activity during feed pelleting remains the most limiting factor for use of phytase as a feed supplement. Temperatures can reach 80° C. or greater during feed pelleting. As such, it is advantageous for a phytase for use in feed supplementation to show resilience to the high temperatures reached during feed pelleting. Efforts to identify thermostable naturally occurring phytases have focused around characterizing phytases iii extremophiles, which are organisms living in environments having extreme heat. Bioinformatical approaches to identify novel phytases with potential thermostability based on publically available sequences have also been used.

Despite these efforts, no naturally occurring plant derived phytase has been identified that is thermostable at the high temperatures achieved during the feed milling process. With this in mind, disclosed herein is a thermostable phytase derived from the roots of the Pteris vittata L. (PV, root PV phytase) useful for improving phosphorus utilization in plants and animals, thereby reducing the amount of phosphorus deposited on land. Also disclosed are compositions, systems, and methods of producing and using root PV phytase for improving phosphorus utilization in plants and animals.

The embodiments of the present disclosure encompass, among others, isolated nucleotide, particularly cDNA sequences, corresponding to a phytase derived from the roots of PV, isolated peptide sequences for root PV phytase, vectors including a root PV phytase gene, vectors including antisense sequences for a root PV phytase gene, vectors for over-expression of a derived PV phytase gene, transgenic and introgressed plants and plant cells that express an exogenous root-derived PV phytase gene, genetically modified bacterial, fungal, and yeast cells expressing an exogenous root PV phytase gene, animal feed including isolated PV phytase of the present disclosure, and microbial fertilizers containing genetically modified bacterial, fungal, and/or yeast cells expressing an exogenous root PV phytase.

As demonstrated by the examples below, one advantage of the root PV phytase disclosed herein can be that it unexpectedly retains 100% of its activity at temperatures of greater than about 70° C., particularly between 70° C. and about 100° C. Further, the root PV phytase can be unaffected by arsenate, a well-known inhibitor of other phytases. Moreover, the root PV phytase can be resistant to deactivation by sorption in soil, unlike other plant phytases.

With this general description and several advantages of the disclosed embodiments in mind, attention is directed to a detailed discussion of the various embodiments described herein.

Nucleic Acid Sequences

Isolated Nucleotide and cDNA Sequences

The present disclosure describes isolated nucleotide and cDNA sequences, which either in whole or in part, can encode a phytase from the roots of PV. In some embodiments, the root PV phytase encoded by an isolated or synthetic nucleotide or cDNA sequence cleaves phosphate from phytate. In one embodiment, the root PV phytase has 50% or greater activity at temperatures of about 70° C. or greater. In some embodiments, the root PV phytase encoded by the isolated nucleotide or cDNA sequence has about 100% activity at temperatures greater than about 70° C., and in one embodiment, about 100% activity at temperatures between 70° C. and about 100° C. In other embodiments, the root PV phytase encoded by the isolated nucleotide or cDNA sequence has greater than about 90% activity at temperatures greater than about 80° C., and in one embodiment, greater than about 90% activity at temperatures between 80° C. and about 100° C. In further embodiments, the root PV phytase encoded by the isolated nucleotide or cDNA sequence has greater than about 25% percent activity at temperatures greater than about 90° C., and in one embodiment, greater than about 25% activity at temperatures between about 90° C. and about 100° C.

In some embodiments, a nucleotide encoding a phytase from the root of PV can have an isolated nucleotide sequence according to SEQ ID NO: 1. In some embodiments, cDNA corresponding to a root PV phytase can have a sequence corresponding to any one SEQ ID NOs: 2, 8, 10, 12, 14, 16, 18, or 20. The cDNA can have a sequence with at least 99% identity to any one of SEQ ID NOs: 2, 8, 10, 12, 14, 16, 18, or 20. In some embodiments, the cDNA can a sequence having at least 98% identity to any one of SEQ ID NOs: 2, 8, 10, 12, 14, 16, 18, or 20. In other embodiments, the cDNA can have a sequence having at least 95%, at least 90%, at least 85%, at least 80%, at least 70%, or at least 50% sequence identity to any one of SEQ ID NOs: 2, 8, 10, 12, 14, 16, 18, or 20. In further embodiments, the cDNA sequence has between about 70% and about 80%, or between about 80% and 90%, or between about 90% and about 100% sequence identity with any one of SEQ ID NOs: 2, 8, 10, 12, 14, 16, 18, or 20.

In additional embodiments, the cDNA has a sequence corresponding to SEQ ID NO: 2 and has between about 70% and about 100% sequence identity with SEQ ID NO: 4. In further embodiments, the cDNA has a sequence corresponding to SEQ ID NO: 2 and has between about 70% and about 100% sequence identity with SEQ ID NO: 6. In other embodiments, the cDNA has a sequence corresponding to SEQ ID NO: 2 and has about 70% and about 100% sequence identity with SEQ ID NO: 8. The cDNA can have a sequence corresponding to SEQ ID NO: 2 and has about 70% and about 100% sequence identity with SEQ ID NO: 10. The cDNA can have a sequence corresponding to SEQ ID NO: 2 and has about 70% and about 100% sequence identity with SEQ ID NO: 12. The cDNA can have a sequence corresponding to SEQ ID NO: 14 and has about 70% and about 100% sequence identity with SEQ ID NO: 10. The cDNA can have a sequence corresponding to SEQ ID NO: 2 and has about 70% and about 100% sequence identity with SEQ ID NO: 16. The cDNA can have a sequence corresponding to SEQ ID NO: 2 and has about 70% and about 100% sequence identity with SEQ ID NO: 18. The cDNA can have a sequence corresponding to SEQ ID NO: 2 and has about 70% and about 100% sequence identity with SEQ ID NO: 20.

In some embodiments, a root PV phytase cDNA encodes a polypeptide having a sequence at least 90% identity to any one of SEQ ID NOs: 3, 5, 7, 9, 11, 13, 15, 17, 19, or 21. In additional embodiments, the root PV phytase cDNA encodes a polypeptide having a sequence between about 90% and about 100% sequence identity to any one of SEQ ID NOs: 3, 5, 7, 9, 11, 13, 15, 17, 19, or 21. In other embodiments, the root PV phytase cDNA encodes a polypeptide having a sequence between about 90% and about 100% sequence identity to any one of SEQ ID NOs: 3, 9, 11, 13, 15, 17, 19, or 21 and between about 70% and about 100% sequence identity to SEQ ID NO: 5. In further embodiments, the cDNA encodes a polypeptide having a sequence between about 90% and about 100% sequence identity to any one of SEQ ID NOs: 3, 9, 11, 13, 15, 17, 19, or 21 and between about 70% and about 100% sequence identity to SEQ ID NO: 7. The cDNA can encode a polypeptide have between about 90% and about 100% sequence identity to any one of SEQ ID NOs: 3, 5, 7, 9, 11, 13, 15, 17, 19, or 21.

The present disclosure also describes isolated nucleotide fragments, including synthetic nucleotide fragments and cDNA fragments, of at least 6 nucleotides sequences having between about 90% and 100%, between about 95% and about 100%, or between about 99% and 100% sequence identity with any sequence within any one of SEQ ID NOs: 1, 2, 8, 10, 12, 14, 16, 18, or 20. In some embodiments, the isolated nucleotide or synthetic nucleotide fragments have about 90% to about 100% sequence identity to any one of SEQ ID NOs: 1, 2, 8, 10, 12, 14, 16, 18, or 20 and about 70% to about 100% sequence identity to SEQ ID NO: 4 and/or SEQ ID NO: 6. Suitable isolated nucleotide or synthetic nucleotide fragments can be obtained by using standard methods known to those of skill in the art, including but not limited to, restriction enzyme digestion and polymerase chain reaction (PCR), or de novo nucleotide sequence synthesis techniques.

In some embodiments, the isolated or synthetic nucleotide fragment encodes a peptide or polypeptide capable of cleaving phosphate from phytate. In one embodiment, the isolated or synthetic nucleotide fragment encodes a peptide or polypeptide having at about 50% or greater activity at temperatures of about 70° C. or greater. In some embodiments, the peptide or polypeptide encoded by the isolated or synthetic nucleotide fragment has about 100% activity at temperatures greater than about 70° C., and in one embodiment, about 100% activity at temperatures between about 70° C. and about 100° C. In other embodiments, the peptide or polypeptide encoded by the isolated or synthetic nucleotide has about 90% activity at temperatures greater than about 80° C., and in one embodiment, greater than about 90% activity at temperatures between about 80° C. and about 100° C. In further embodiments, the peptide or polypeptide encoded by the isolated or synthetic nucleotide fragment has about 25% percent activity at temperatures greater than about 90° C., and in one embodiment, greater than about 25% activity at temperatures between about 90° C. and about 100° C.

In other embodiments, the present disclosure includes isolated or synthetic antisense polynucleotides capable of inhibiting expression of an endogenous root PV phytase gene. The polynucleotides that are capable of inhibiting expression of the root PV phytase gene may inhibit expression directly (e.g., by binding to the root PV phytase mRNA to prevent translation) or via a transcription product (e.g., RNA if the antisense polynucleotide is DNA) of the antisense polynucleotide. Such antisense polynucleotides can be used in vectors to produce transgenic plant varieties or cell lines where root PV phytase expression is inhibited or down-regulated, thus reducing root PV phytase in the transgenic plant or cell line. In some embodiments, the antisense polynucleotides of the present disclosure are capable of inhibiting expression of an endogenous root PV phytase gene whose cDNA corresponds to any one of SEQ ID NOs: 2, 8, 10, 12, 14, 16, 18, or 20 or having at least about 90% sequence identity with any one of SEQ ID NOs: 1, 2, 8, 10, 12, 14, 16, 18, or 20. In other embodiments, the antisense polynucleotides have between about 90% and about 100%, or between about 95% and about 100%, or between about 99% and 100% sequence identity with any one of SEQ ID NOs: 2, 8, 10, 12, 14, 16, 18, or 20. In some embodiments, when the antisense polynucleotides of the present disclosure are transcribed in a plant or cell line, such antisense polynucleotides can inhibit expression of an endogenous or exogenous root-derived PV phytase gene whose cDNA corresponds to any one of SEQ ID NOs: 2, 8, 10, 12, 14, 16, 18, or 20 or having between about 90% and 100% sequence identity with any one of SEQ ID NOs: 2, 8, 10, 12, 14, 16, 18, or 20.

Recombinant Polynucleotide Sequences

The present disclosure also includes recombinant polynucleotide sequences having any of the isolated nucleotide or cDNA sequences or fragments thereof previously described and additional polynucleotide sequences operatively linked to the isolated nucleotide or cDNA sequences or fragments thereof. In some embodiments, non-coding nucleotides can be placed at the 5′ and/or 3′ end of the polynucleotides encoding root PV phytase peptides or the antisense polynucleotides without affecting the functional properties of the molecule. A polyadenylation region at the 3′-end of the coding region of a polynucleotide can be included. The polyadenylation region can be derived from the endogenous gene, from a variety of other plant genes, from T-DNA, or through chemical synthesis. In further embodiments, the nucleotides encoding the root PV phytase polypeptide may be conjugated to a nucleic acid encoding a signal or transit (or leader) sequence at the N-terminal end (for example) of the root PV phytase polypeptide that co-translationally or post-translationally directs transfer of the root PV phytase polypeptide. The polynucleotide sequence may also be altered so that the encoded root PV phytase polypeptide is conjugated to a linker, selectable marker, or other sequence for ease of synthesis, purification, and/or identification of the protein. In one embodiment, the recombinant polynucleotide sequence includes at least one regulatory sequence operatively linked to the isolated nucleotide or cDNA sequences or fragments thereof.

To express an exogenous root PV phytase gene, fragment thereof, or antisense nucleotide in a cell, the exogenous nucleotide can be combined (e.g., in a vector) with transcriptional and/or translational initiation regulatory sequences, i.e. promoters, that direct the transcription of the gene and/or translation of the encoded protein in a cell. In some embodiments a constitutive promoter may be employed. Suitable constitutive promoters for plant cells include, but are not limited to, the cauliflower mosaic virus (CaMV) 35S transcription initiation region, the 1′- or 2′-promoter derived from T-DNA of Agrobacterium tumefaciens, the ACT11 and Cat3 promoters from Arabidopsis (Huang et al. Plant Mol. Biol. 1996, 33:125-139 and Zhong et al. Mol. Gen. Genet. 1996, 251:196-203), the stearoyl-acyl carrier protein desaturase gene promoter from Brassica napus (Solocombe et al. Plant Physiol. 1994, 104:1167-1176), and the GPc 1 and Gpc2 promoters from maize (Martinez et al. J. Mol. Biol. 1989, 208:551-565 and Manjunath et al. Plant Mol. Biol. 1997, 33:97-112). Suitable constitutive promoters for bacterial cells, yeast cells, fungal cells are generally known in the art, such as a T-7 promoter for bacterial expression and an alcohol dehydrogenase promoter for expression in yeast.

In other embodiments, tissue-specific promoters or inducible promoters may be employed to direct expression of the exogenous nucleic acid in a specific cell type, under certain environmental conditions, and/or during a specific state of development. Examples of environmental conditions that may affect transcription by inducible promoters include anaerobic conditions, elevated temperature, the presence of light, contact with chemicals or hormones, or infection by a pathogen. Suitable plant inducible promoters include the root-specific ANRI promoter (Zhang and Forde. Science. 1998, 279:407), the photosynthetic organ-specific RBCS promoter (Khoudi et al. Gene. 1997, 197:343) and the tomato fruit ripening-specific E8 promoter (Deikman, J., et al. Plant Physiol. 1992, 100: 2013-2017).

A selectable marker can also be included in the recombinant nucleic acid to confer a selectable phenotype on plant cells. For example, the selectable marker may encode a protein that confers biocide resistance, antibiotic resistance (e.g., resistance to kanamycin, G418, bleomycin, hygromycin), or herbicide resistance (e.g., resistance to chlorosulfuron or Basta). Thus, the presence of the selectable phenotype indicates the successful transformation of the host cell. An exemplary selectable marker includes the beta-glucuronidase (GUS) reporter gene.

Suitable recombinant polynucleotides can be obtained by using standard methods known to those of skill in the art, including but not limited to, restriction enzyme digestion, PCR, ligation, and cloning techniques. In some embodiments, the recombinant polynucleotide encodes a peptide or polypeptide capable of cleaving phosphate from phytate. In one embodiment, a recombinant polynucleotide of the present disclosure encodes a peptide or polypeptide having about 50% or greater activity at temperatures of about 70° C. or greater. In other embodiments, the recombinant polynucleotide encodes a peptide or polypeptide that has about 100% activity at temperatures greater than about 70° C., and in one embodiment, about 100% activity at temperatures between 70° C. and about 100° C. In further embodiments, the recombinant polynucleotide encodes a peptide or polypeptide that has about 90% activity at temperatures greater than about 80° C., and in one embodiment, greater than about 90% activity at temperatures between about 80° C. and about 100° C. In still other embodiments, the recombinant polynucleotide encodes a peptide or polypeptide that has about 100% percent activity at temperatures greater than about 90° C., and in one embodiment, greater than about 90% activity at temperatures between about 90° C. and about 100° C.

Isolated Protein (Polypeptide) and Peptide Sequences:

The present disclosure also describes an isolated or synthetic protein (polypeptide) corresponding to a phytase from the roots of PV. In some embodiments, the isolated polypeptide has an amino acid sequence corresponding to any one of SEQ ID NOs: 3, 9, 11, 13, 15, 17, 19, or 21. SEQ ID NO: 3 is an amino acid sequence of a phytase derived from the root of Pteris vittata L. SEQ ID NOs: 9, 11, 13, 15, 17, 19, or 21 are predicted to correspond to root PV purple acid phytases. The isolated or synthetic polypeptide can have an amino acid sequence with at least about 99% identity to any one of SEQ ID NOs: 3, 9, 11, 13, 15, 17, 19, or 21. In some embodiments, the isolated or synthetic polypeptide has an amino acid sequence having at least about 98% identity to any one of SEQ ID NOs: 3, 9, 11, 13, 15, 17, 19, or 21. In other embodiments, the isolated polypeptide has an amino acid sequence having at least about 95%, at least about 90%, at least about 85%, at least about 80%, at least about 70%, or at least about 50% sequence identity to any one of SEQ ID NOs: 3, 9, 11, 13, 15, 17, 19, or 21. In some embodiments, the isolated or synthetic polypeptide has greater than about 70%, or between about 70% and about 90%, or between about 90% and 100% sequence identity to any one of SEQ ID NOs: 3, 9, 11, 13, 15, 17, 19, or 21. In one embodiment, the isolated or synthetic polypeptide has about 80% to about 100% sequence identity to Glycine Max phytase (SEQ ID NO: 5) and/or Medicago truncatula (SEQ ID NO: 7).

In some embodiments the isolated or synthetic polypeptide as disclosed herein cleaves phosphate from phytate. In one embodiment, the isolated polypeptide has about 50% or greater activity at temperatures of about 70° C. or greater. In some embodiments, the isolated or synthetic polypeptide has about 100% activity at temperatures greater than about 70° C., and in one embodiment, about 100% activity at temperatures between about 70° C. and about 100° C. In other embodiments, the isolated or synthetic polypeptide has about 90% activity at temperatures greater than about 80° C., and in one embodiment, greater than about 90% activity at temperatures between about 80° C. and about 100° C. In further embodiments, the isolated or synthetic polypeptide has about 25% percent activity at temperatures greater than about 90° C., and in one embodiment, greater than about 25% activity at temperatures between about 90° C. and about 100° C.

Modifications and changes can be made in the structure of the polypeptides of the present disclosure that result in a molecule having similar characteristics as the unmodified polypeptide (e.g., a conservative amino acid substitution). Modification techniques are generally known in the art. For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a functional variant. Polypeptides with amino acid sequence substitutes that still retain properties substantially similar to polypeptides corresponding to root PV phytase are within the scope of this disclosure.

The present disclosure also includes isolated and synthetic peptides corresponding to a fragment of the polypeptide corresponding to root PV phytase. In some embodiments the peptides correspond to a portion of any one of SEQ ID NOs: 3, 9, 11, 13, 15, 17, 19, or 21. The isolated or synthetic peptides have at least about 90%, or at least about 95%, or at least about 99% sequence identity to any portion of any one of SEQ ID NOs: 3, 9, 11, 13, 15, 17, 19, or 21. In some embodiments, the isolated or synthetic peptides have between about 90% and about 95%, or between about 95% and about 99%, or between about 99% and about 100% sequence identity to a sequence within any one of SEQ ID NOs: 3, 9, 11, 13, 15, 17, 19, or 21. In some embodiments, the isolated peptides have between about 70% and 100% sequence identity with a portion of Glycine max phyase (SEQ ID NO: 5) and/or M. truncatula (SEQ ID NO: 7).

In some embodiments, the isolated peptide can cleave phosphate from phytate. In one embodiment, the isolated or synthetic peptide can have at least about 50% activity at temperatures of about 70° C. or greater. In some embodiments, the isolated or synthetic peptide has about 100% activity at temperatures greater than about 70° C., and about 100% activity at temperatures between about 70° C. and about 100° C. In other embodiments, the isolated peptide or synthetic peptide has about 90% activity at temperatures greater than about 80° C., and greater than about 90% activity at temperatures between 80° C. and about 100° C. In further embodiments, the isolated or synthetic peptide has about 25% percent activity at temperatures greater than about 90° C., greater than about 25% activity at temperatures between about 90° C. and about 100° C.

In other embodiments, the isolated or synthetic peptide as described herein is suitable for use in production of antibodies against root PV phytase. In other words, the isolated or synthetic peptide as described herein serves as the antigen to which an antibody is raised against. In some embodiments, the isolated or synthetic peptide sequence is also the epitope of the antibody. Antibodies raised against root-PV phytase are suitable for use in methods for at least detection, quantification, and purification of root PV phytase. Other uses for anti-root PV phytase antibodies are generally known in the art.

Vectors

Vectors having one or more of the polynucleotides or antisense polynucleotides described herein can be useful in producing transgenic bacterial, fungal, yeast, plant cells, and transgenic plants that express varying levels of a root PV phytase. Within the scope of this disclosure are vectors containing one or more of the polynucleotide sequences described herein.

In one embodiment, the vector includes a polynucleotide encoding a root PV phytase, where the DNA molecule has at least about 90%, or between about 90% and about 95%, or between about 95% and about 100% sequence identity with any one of SEQ ID NOs: 2, 8, 10, 12, 14, 16, 18, or 20. In some embodiments, the vector includes a DNA molecule encoding a root PV phytase, where the DNA molecule has at least about 90%, or between about 90% and about 95%, or about 95% and about 100% sequence identity with any one of SEQ ID NOs: 2, 8, 10, 12, 14, 16, 18, or 20, and where the root PV phytase catalyzes removal of phosphate from phytate. In further embodiments, the vector includes a DNA molecule encoding a root PV phytase, where the DNA molecule has at least about 90%, or between about 90% and about 95%, or between about 95% and about 100% sequence identity with any one of SEQ ID NOs: 2, 8, 10, 12, 14, 16, 18, or 20, and where the root PV phytase catalyzes removal of phosphate from phytate at temperatures between about 70° C. and about 80° C., or between about 80° C. and about 90° C., or between about 90° C. and about 100° C. In other embodiments, the vector includes a DNA molecule encoding a root PV phytase, where the DNA molecule has at least about 90%, or between about 90% and about 95%, and or between about 95% and about 100% sequence identity with any one of SEQ ID NOs: 2, 8, 10, 12, 14, 16, 18, or 20, and where the root PV phytase catalyzes removal of phosphate from phytate at temperatures greater than about 100° C. In some embodiments, the vector has a cDNA molecule that encodes a polypeptide having a sequence with at least about 90%, or between about 90% and about 95%, or between 95% and about 100% sequence identity to any one of SEQ ID NOs: 3, 9, 11, 13, 15, 17, 19, or 21.

In one embodiment, the vector has at least one regulatory sequence operatively linked to a DNA molecule or encoding a root PV phytase such that the root PV phytase is expressed in a bacteria, fungus, yeast, plant, or other cell into which it is transformed. In other embodiments, the vector includes a promoter that serves to initiate expression of the root PV phytase such that the root PV phytase is over-expressed in a plant cell into which it is transformed relative to a wild-type bacteria, fungus, yeast, or plant cell. In some embodiments, the vector has at least one regulatory sequence operatively linked to a DNA molecule encoding a root PV phytase and a selectable marker.

Other embodiments of the present disclosure include a vector having an antisense polynucleotide capable of inhibiting expression of an endogenous the root PV phytase gene and at least one regulatory sequence operatively linked to the antisense polynucleotide such that the antisense polynucleotide is transcribed in a type bacteria, fungus, yeast, or plant cell into which it is transfected. In embodiments, the antisense polynucleotides may be capable of inhibiting expression of an endogenous root PV phytase gene corresponding to any one of SEQ ID NOs: 2, 8, 10, 12, 14, 16, 18, or 20 or at least about 90% sequence identity with any one of SEQ ID NOs: 2, 8, 10, 12, 14, 16, 18, or 20.

Transgenic Plants

The polynucleotide sequences and vectors described above can be used to produce transgenic plants. The present disclosure includes transgenic plants having one or more cells where the one or more cells contain any of the recombinant polynucleotides or vectors previously described that have DNA sequences encoding the root PV phytase. In one embodiment, the recombinant polynucleotide contains at least one regulatory element operatively linked to a root PV DNA sequence having at least about 90%, or between about 90% and about 95%, or between about 95% and about 100% sequence identity with any one of SEQ ID NOs: 2, 8, 10, 12, 14, 16, 18, or 20.

Also described herein are transgenic plants having one or more cells transformed with vectors containing any of the nucleotide sequences described above, and/or fragments of the nucleic acids encoding the root PV phytase proteins of the present disclosure. In some embodiments, the vector contains a root PV DNA sequence having at least about 90%, or between about 90% and about 95%, or between about 95% and about 100% sequence identity with any one of SEQ ID NOs: 2, 8, 10, 12, 14, 16, 18, or 20. The transgenic plant can be made from any suitable plant species or variety including, but not limited to Arabidopsis, rice, wheat, corn, maize, tobacco, soybean, Brassicas, tomato, potato, alfalfa, sugarcane, and sorghum.

In some embodiments, the transgenic plant having a nucleotide sequence encoding root PV phytase has increased expression of root PV phytase relative to a wild type plant. In other embodiments, the transgenic plant has a nucleotide sequence encoding root PV phytase has increased expression of root PV phytase relative to a wild type plant and produces a PV phytase that is capable of cleaving phosphate from phytate. In further embodiments the transgenic plant has a nucleotide sequence encoding root PV phytase has increased expression of root PV phytase relative to a wild type plant.

In some embodiments, the transgenic plant produces a root PV phytase that cleaves phosphate from phytate. In one embodiment, the transgenic plant produces a root PV phytase that has about 50% or greater activity at temperatures of about 70° C. or greater. In some embodiments, the transgenic plant produces a root PV phytase that has about 100% activity at temperatures greater than about 70° C., and about 100% activity at temperatures between about 70° C. and about 100° C. In other embodiments, the transgenic plant produces a root PV phytase that has about 90% activity at temperatures greater than about 80° C. In some embodiments, the transgenic plant produces a root PV phytase that has greater than about 90% activity at temperatures between about 80° C. and about 100° C. In further embodiments, the transgenic plant produces a root PV phytase that has about 25% percent activity at temperatures greater than about 90° C. In some embodiments, the transgenic plant produces a root PV phytase that has greater than about 25% activity at temperatures between about 90° C. and about 100° C.

Similarly, the present disclosure includes transgenic plants having one or more cells where the one or more cells contain any of the recombinant polynucleotides or vectors of the present disclosure previously described that have an antisense DNA sequence capable of decreasing expression of root PV phytase RNA or protein. In one embodiment, the recombinant polynucleotide contains at least one regulatory element operatively linked to an antisense DNA sequence capable of decreasing expression of root PV phytase RNA or protein. Also encompassed by the present disclosure are transgenic plants having one or more cells transformed with vectors containing an antisense DNA sequences capable of decreasing expression of root PV phytase RNA or protein. In some embodiments, the transgenic plant having an antisense DNA sequence capable of decreasing expression of root PV phytase RNA or protein has reduced root PV phytase relative to a wild type plant.

A transformed plant cell of the present disclosure can be produced by introducing into a plant cell on or more vectors as previously described. In one embodiment, transgenic plants of the present disclosure can be grown from a transgenic plant cell transformed with one or more of the vectors previously described. In one embodiment, the cells are transformed with a vector including a recombinant polynucleotide encoding a root PV phytase having at least about 90%, or between about 90% and about 95%, or between about 95% and about 100% sequence identity with any one of SEQ ID NOs: 2, 8, 10, 12, 14, 16, 18, or 20 that has at least one regulatory sequence operatively linked to the DNA molecule.

Techniques for transforming a wide variety of plant cells with vectors or naked nucleic acids are well known in the art and described in the technical and scientific literature. See, for example, Weising et al. Ann. Rev. Genet. 1988, 22:421-477. For example, the vector or naked nucleic acid may be introduced directly into the genomic DNA of a plant cell using techniques such as, but not limited to, electroporation and microinjection of plant cell protoplasts, or the recombinant nucleic acid can be introduced directly to plant tissue using ballistic methods, such as DNA particle bombardment.

Microinjection techniques are known in the art and well described in the scientific and patent literature. The introduction of a recombinant nucleic acid using polyethylene glycol precipitation is described in Paszkowski et al. EMBO J. 1984, 3:2717-2722. Electroporation techniques are described in Fromm et al. Proc. Natl. Acad. Sci. USA. 1985, 82:5824. Ballistic transformation techniques are described in Klein et al. Nature. 1987, 327:70-73. The recombinant nucleic acid may also be combined with suitable T-DNA flanking regions and introduced into a conventional Agrobacterium tumefaciens host vector, or other suitable vector. The virulence functions of the Agrobacterium tumefaciens host will direct the insertion of the recombinant nucleic acid including the exogenous nucleic acid and adjacent marker into the plant cell DNA when the cell is infected by the bacteria. Agrobacterium tumefaciens-mediated transformation techniques, including disarming and use of binary vectors, are known to those of skill in the art and are well described in the scientific literature. See, for example, Horsch et al. Science. 1984, 233:496-498; Fraley et al. Proc. Natl. Acad. Sci. USA. 1983, 80:4803; and Gene Transfer to Plants, Potrykus, ed., Springer-Verlag, Berlin, 1995.

A further method for introduction of the vector or recombinant nucleic acid into a plant cell is by transformation of plant cell protoplasts (stable or transient). Plant protoplasts are enclosed only by a plasma membrane and will therefore more readily take up macromolecules like exogenous DNA. These engineered protoplasts can be capable of regenerating whole plants. Suitable methods for introducing exogenous DNA into plant cell protoplasts include electroporation and polyethylene glycol (PEG) transformation. Following electroporation, transformed cells are identified by growth on appropriate medium containing a selective agent.

The presence and copy number of the exogenous nucleic acid in a transgenic plant can be determined using methods well known in the art, e.g., Southern blotting analysis. Expression of the exogenous root PV phytase nucleic acid or antisense nucleic acid in a transgenic plant may be confirmed by detecting an increase or decrease of mRNA or the root PV phytase polypeptide in the transgenic plant. Methods for detecting and quantifying mRNA or proteins are well known in the art.

Transformed plant cells that are derived by any of the above transformation techniques, or other techniques now known or later developed, can be cultured to regenerate a whole plant. In embodiments, such regeneration techniques may rely on manipulation of certain phytohormones in a tissue culture growth medium, typically relying on a biocide or herbicide selectable marker that has been introduced together with the exogenous nucleic acid. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. Plant Phys. 1987, 38:467-486.

Once the exogenous root PV phytase nucleic acid or antisense nucleic acid has been confirmed to be stably incorporated in the genome of a transgenic plant, it can be introduced into other plants by sexual crossing. Any of a number of standard breeding techniques can be used, depending upon the species to be crossed.

In some embodiments, the seeds or other parts of the plant obtained from transgenic plants expressing root PV phytase made according to the present disclosure are included in an animal feed. For example, kernels of transgenic corn expressing root PV phytase can be used directly to produce animal feed containing root PV phytase. In other embodiments, the seeds or other parts of the plant obtained from plants carrying an allelic variant of root PV phytase, either naturally or by selective breeding techniques, are used as ingredients for the production of animal feed. Animal feeds containing a component of a plant either naturally expressing or genetically modified to express root PV phytase can then be fed to animals.

Genetic Markers

The present disclosure also includes genetic markers useful for identifying different alleles of the root PV phytase gene in other plant varieties and species. In embodiments, such markers may include, but are not limited to restriction fragment length polymorphisms (RFLP), amplified fragment length polymorphisms (AFLP), single nucleotide polymorphisms (SNPs), microsatellite markers (e.g., SSRs), sequence-characterized amplified region (SCAR) markers, variable number tandem repeats (VNTR), short tandem repeats (STR), cleaved amplified polymorphic sequence (CAPS) markers, and isozyme markers, and similar markers or combinations of such markers for the root PV phytase gene. Primers to identify the nucleotide sequence include forward 5′-CCT TGG CAA GCT CAA GAC CA-3′ (SEQ ID NO: 22) and reverse 5′-ATG GAC ATG GCC AGC AAA CA-3,′ (SEQ ID NO: 23) which encodes a 400 bp nucleotide strand of root PV DNA.

Introgression Lines

In some embodiments, homologous alleles or variants of the root PV phytase can be identified in commercially relevant plants with the use of genetic markers of the present disclosure. These homologous or variant alleles can be characterized, and the alleles responsible for the desired heat and arsenic tolerant phytase expression can be identified. In embodiments of the present disclosure, new commercially relevant plant varieties can be obtained by introgressing the desired alleles conferring heat and arsenic tolerant phytase activity. Introgression can be marker-assisted introgression. Breeding techniques to introgress genes and chromosomal segments from one plant variety containing desired alleles are generally known in the art.

Transformed Cells

This disclosure also encompasses one or more cells transformed with one or more isolated nucleotide or cDNA sequences and/or vectors as previously described. In some embodiments, the transformed cell is a plant, bacterial, fungal, or yeast cell. In one embodiment, a plant, bacterial, fungal or yeast cell contains one or more vectors as previously described. Also, within the scope of this disclosure are populations of cells where about 1% to about 100%, or between about 50% and about 75%, or between about 75% and about 100% of the cells within the population contain a vector as previously described.

In some embodiments, one or more cells within the population contain more than one type of vector. In some embodiments, all (about 100%) the cells that contain a vector have the same type of vector. In other embodiments, not all the cells that contain a vector have the same type of vector or plurality of vectors. In some embodiments, about 1% to about 100%, or between about 50% and about 75%, or between about 75% and about 100% of the cells within the population contain the same vector or plurality of vectors. In some cell populations, all the cells are from the same species. Other cell populations contain cells from different species. Transfection methods for establishing transformed (transgenic) cells are well known in the art.

In one embodiment, the transformed cells produce a peptide or polypeptide that cleaves phosphate from phytate. In one embodiment, the transformed cells produce a root PV phytase that has about 50% or greater activity at temperatures of about 70° C. or greater. In some embodiments, the transformed cells produce a peptide or polypeptide that has about 100% activity at temperatures greater than about 70° C., and about 100% activity at temperatures between about 70° C. and about 100° C. In other embodiments, the transformed cells produce a peptide or polypeptide that has about 90% activity at temperatures greater than about 80° C., and greater than about 90% activity at temperatures between about 80° C. and about 100° C. In further embodiments, the transformed cells produce a peptide or polypeptide that has about 25% percent activity at temperatures greater than about 90° C. In another embodiment, the transformed cells produce a peptide or polypeptide that has greater than about 25% activity at temperatures between about 90° C. and about 100° C.

Vector, Polypeptide, and Microbial Fertilizers

In some embodiments, a vector or vectors, as previously described herein, are used as a fertilizer to enhance phytate utilization by plants. In one embodiment, the vector or vectors are mixed with a soil at any suitable concentration. In further embodiments, the vector or vectors are mixed with the soil surrounding the root tips of plants in the soil.

In other embodiments, a purified root PV phytase or isolated root PV polypeptide as described herein, are used as a fertilizer to enhance phytate utilization by plants. In one embodiment the purified root PV phyatse or isolated root PV polypeptide are mixed with a soil at any suitable concentration. In further embodiments, the purified root PV phytase or isolated root PV polypeptide are mixed with the soil surrounding the root tips of plants.

In further embodiments, transformed cells, as previously described herein, can be used as a microbial fertilizer. In some embodiments, the transformed cells are included in a composition that is used as a microbial fertilizer. The microbial fertilizer can include a cell population wherein about 1% to about 100%, or between about 50% and about 75%, or between about 75% and about 100% of the cells are transformed and include one or more of the types of vectors previously described. In one embodiment, the transformed cells contain a vector having a cDNA with at least about 90%, or between about 90% and about 95%, or between about 95% and about 100% sequence identity to any one of SEQ ID NOs: 2, 8, 10, 12, 14, 16, 18, or 20.

In some embodiments, all cells within the cell population are transformed with the same type vector or vectors. In other embodiments, some cells are transformed with different types of vector or vectors than other cells creating a mixed cell population. The cells used in the microbial fertilizer can be any suitable cell, including bacteria, fungal, yeast cells, or plant cells. The microbial fertilizer can be added to soil at any suitable concentration. In one embodiment, the microbial fertilizer is added to the soil surrounding the root tips of plants in the soil.

Purified Recombinant Root PV Phytase

The present disclosure also encompasses a purified recombinant root PV phytase that is used as a feed additive or supplement for animal feeds. Purified recombinant root PV phytase can be purified from cells transformed as previously described and as further discussed below. The purified recombinant root PV phytase made according to this disclosure can be further modified to optimize utilization by an animal. In some embodiments, the purified recombinant root PV phytase has a primary amino acid sequence having at least about 90%, or between about 90% and about 95%, or between about 95% and about 100% sequence identity to any one of SEQ ID NOs: 3, 9, 11, 13, 15, 17, 19, or 21.

In one embodiment, the purified recombinant root PV phytase cleaves phosphate from phytate. In one embodiment, the purified recombinant root PV phytase has about 50% or greater activity at temperatures of about 70° C. or greater. In some embodiments, the purified recombinant root PV phytase has about 100% activity at temperatures greater than about 70° C. In other embodiments, the purified recombinant root PV phytase has about 100% activity at temperatures between about 70° C. and about 100° C. In further embodiments, the purified recombinant root PV phytase has about 90% activity at temperatures greater than about 80° C., and in other embodiments, greater than about 90% activity at temperatures between about 80° C. and about 100° C. In another embodiment, the purified recombinant root PV phytase has about 25% percent activity at temperatures greater than about 90° C. In further embodiments, purified recombinant root PV phytase has greater than about 25% activity at temperatures between about 90° C. and about 100° C.

In some embodiments, the purified recombinant root PV phytase is coated with a suitable coating to optimize stability, enhance digestibility, or to otherwise optimize the activity of the recombinant root PV phytase within the animal. Purified recombinant root PV phytase can be added to feed at any stage during the milling process. The feed containing the purified recombinant root PV phytase can then be fed to animals.

In embodiments, to produce purified recombinant root PV phytase, transformed cells having an isolated nucleotide, cDNA, and/or vector encoding a recombinant root PV phytase, as described herein, are grown in cultures and the recombinant root PV phytase produced in culture is then purified from the cell culture components according to methods generally known in the art. The cultures can be scaled and modified accordingly by methods known in the art to produce the purified recombinant root PV phytase on any scale.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

### Cloning of a Root-Derived PV Phytase

### Materials and Methods

Total RNA was extracted from fresh root tips of Pteris vittata grown in nutrient solution amended with phytic acid in lieu of soluble phosphate. Total RNA was isolated using the Spectrum™ Plant Total RNA Kit (Sigma-Aldrich). cDNA was synthesized using a 2Step RT-PCR Kit (Qiagen). The DNA encoding phytase was amplified by PCR from cDNA using

the degenerate forward primer 5′-GGN GAY YTI GGN CAR AC-3′ (P1) (SEQ ID NO: 24) and reverse primer 5′-TGC CAI SWC CAR TGN GCR TG-3′ (P2) (SEQ ID NO: 25) designed from areas of high sequence homology of Selaginella moellendorffii (NCBI Accession No. XP_002981872.1) to other well characterized plant phytases.

The reaction system for reverse transcription included 4 μL of RT buffer (Qiagen), 2 μL dNTP (10 mM), 1 μL Oligo-dT (20 μM), 0.2 μL RNase inhibitor, 1 μL reverse transcriptase, 2 μg of template RNA, and RNase-free water for a final volume of 20 μL. Samples were incubated for 90 min at 42° C. followed by 5 min at 85° C. to inactivate the enzyme.

The PCR amplification reaction system was composed of 5 μL of PCR buffer with Mg²⁺ (Qiagen), 2.5 μL dNTP (10 mM), 1 μL of forward and reverse primers (0.4 μM), 0.4 μl PCR enzyme mix, 3 μL of template cDNA, and RNase-free water for a final volume of 50 pt. Samples were placed in a PCR cycler (Thermo-Scientific) with the following conditions: initial denaturation at 95° C. for 5 min, denaturation at 95° C. for 0.25 min, annealing temperature at 56° C. for 0.5 min, extension at 72° C. for 2 min, final extension at 72° C. for 5 min. Samples were run on a 0.8% agarose gel, cutting individual bands for DNA purification using a DNA Clean and Concentrator Kit (Zymo). Samples were sent to the Interdisciplinary Center for Biotechnology Research (ICBR) at the University of Florida for Sanger analysis to determine nucleotide sequence. Amplified sequences contained >70% homology to >50 plant phytases using a nucleotide query in NCBI BLAST.

Amplification of the 5′ and 3′ ends of the Pteris vittata sequence were conducted using a 5′/3′ RACE kit, 2^{nd} Generation (Roche). Primers were developed from the partial sequence: forward primer 5′-CCT TGG CAA GCT CAA GAC CA-3′ (P3) (SEQ ID NO: 26), reverse primer 5′-ATG GAC ATG GCC AGC AAA CA-3′ (P4) (SEQ ID NO: 27) and reverse primer 5′-GCC AAA TCA GCC AGA AGC CA-3′ (P5) (SEQ ID NO: 28). For identification of the 3′, first strand cDNA synthesis was performed by adding 4 μL of cDNA Synthesis buffer (Roche), 2 μL dNTP (10 mM), 1 μL Oligo-dT-Anchor Primer (Roche), 1 μL reverse transcriptase, 2 μg of template RNA, and RNase-free water for a final volume of 20 μL. Samples were incubated for 60 min at 55° C. followed by 5 min at 85° C. to inactivate the enzyme. Next, 1 μL of the cDNA product were combined with 1 μL of PCR Anchor Primer, 1 μL dNTP (10 mM), 1 μL of primer P3, 0.75 μl Expand high fidelity enzyme mix, 5 μl Expand high fidelity buffer with 15 mM MgCl2 and RNase-free water for a final volume of 50 μL. Samples were placed in a PCR cycler (Thermo-Scientific) with the following conditions: initial denaturation at 95° C. for 5 min, denaturation at 95° C. for 0.25 min, annealing temperature at 60° C. for 0.5 min, extension at 72° C. for 2 min, final extension at 72° C. for 5 min. Samples were run on a 0.8% agarose gel, cutting individual bands for DNA purification using a DNA Clean and Concentrator Kit (Zymo). Samples were sent to the Interdisciplinary Center for Biotechnology Research (ICBR) at the University of Florida for Sanger analysis to determine nucleotide sequence. Amplified sequences contained >70% homology to >50 plant phytases using a nucleotide query in NCBI BLAST.

For identification of the 5′ end, first strand cDNA synthesis was performed by adding 4 μL of cDNA Synthesis buffer (Roche), 2 μL dNTP (10 mM), 1 μL of primer P4, 1 μL reverse transcriptase, 2 μg of template RNA, and RNase-free water for a final volume of 20 μL. Samples were incubated for 60 min at 55° C. followed by 5 min at 85° C. to inactivate the enzyme. Samples purified using a DNA Clean and Concentrator Kit (Zymo). Poly(A) tailing of the cDNA sample was achieved by combining 19 μL of the cDNA sample, 2.5 μL of reaction buffer (Roche), 2.5 μL dATP (2 mM) and incubating for 3 min at 94° C. followed by being chilled on ice. 1 μL of terminal transferase was then added and samples incubated at 37° C. for 30 min and 70° C. for 10 min.

Next, 5 μL of the dA-tailed cDNA product were combined with 1 μL of Oligo dT-Anchor Primer (Roche), 1 μL dNTP, 1 μL of primer P5, 0.75 μL Expand high fidelity enzyme mix, 5 μl Expand high fidelity buffer with 15 mM MgCl₂ and RNase-free water for a final volume of 50 μL. Samples were placed in a PCR cycler (Thermo-Scientific) with the following conditions: initial denaturation at 95° C. for 5 min followed by 25 cycles of denaturation at 95° C. for 0.25 min, annealing temperature at 60° C. for 0.5 min, extension at 72° C. for 2 min, and a final extension at 72° C. for 5 min. Samples were run on a 0.8% agarose gel, cutting individual bands for DNA purification using a DNA Clean and Concentrator Kit (Zymo). Samples were sent to the Interdisciplinary Center for Biotechnology Research (ICBR) at the University of Florida for Sanger analysis to determine nucleotide sequence. Amplified sequences contained >70% homology to >50 plant phytases using a nucleotide query in NCBI BLAST. Overlapping regions of the 3′ and 5′ regions confirmed, containing >99% sequence homology. The sequence was translated into amino acid sequence using bioinformatics software ExPASy and tblastx in NCBI BLAST. The cDNA region was confirmed by comparing the amino acid sequence against other plant phytases using NCBI B. Amplified sequences contained >70% homology to >50 plant phytases using a protein query in NCBI BLAST.

### Results:

A root PV phytase cDNA (SEQ ID NO: 2) has 66% sequence identity with Glycine Max clone GMPhy03 mRNA (>gb|GQ422771.1) (which corresponds to cDNA SEQ ID NO: 4) with 79% query coverage. The root PV phytase cDNA has 66% sequence identity with purple acid phosphatase (Medicago truncatula) mRNA (MTR_5g092360) (which corresponds to SEQ ID NO: 6) with 72% query coverage. The amino acid sequence of Root PV phytase has 60% sequence identity with the amino acid sequence of purple acid phosphatase polypeptide (Medicago truncatula) (SEQ ID NO: 7) (>gb|AES70308.1) with 85% query coverage. The amino acid sequence of Root PV phytase has 60% sequence identity with the amino acid sequence of purple acid phosphatase polypeptide (Glycine Max) (>gb|ADM32490.1) (SEQ ID NO: 5) with 92% query coverage. Query coverage is the percent of the query sequence that overlaps the subject sequence.

### Example 2

### Resistance of Root-derived PV Phytase to Arsenic

### Methods:

Enzyme Collection.

Root Tissues from P. Vittata (PV) and P. enisformis (PE) were rinsed in 10 mM Ca(NO₃)₂ and blotted dry, weighed, and mixed (1:2 w/v) with 10 mM acetate buffer (pH 5.0) containing 1 mM EDTA, 1 mM DTT (dithiothreitol), 0.1 mM PMSF (phenylmethylsulfonyl fluoride), and 4% PVPP (polyvinyl polypyrrolidone). Samples were homogenized using a Magic Bullet® blender (Four 15 s pulses), passed through cheesecloth and centrifuged at 10,000 g for 15 min. Supernatants were subjected to ammonium sulfate fractionation, collecting precipitates in 20% intervals from 0-80% fractions followed by gel filtration on Sephadex G-25, which was pre-equilibrated with 10 mM acetate buffer (pH 5.0). Root exudates were collected from media of 40 d old PV sporophyte, analyzing enzyme activity following previously described purification steps.

Phytase and Phosphatase Assays.

Protein content was measured against bovine serum albumin (BSA) standards using the Bradford method (Walker et al., The Bradford Method for Protein Quantitation. In The Protein Protocols Handbook; Humana Press, 2002; pp. 15-21). Enzyme activity was analyzed by incubating ˜100 μg protein in 1 mL of 10 mM acetate buffer (pH 5.0) containing either 5 mM phytate or 5 mM pNPP (p-nitrophenylphosphate disodium salt; Sigma) at 37° C. for phytase and phosphatase, respectively. Reactions were terminated with equal volumes of 10% (wt/v) trichloroacetic acid after 120 min (phytase) or 25 mM NaOH after 30 min (phosphatase). Specific activities were calculated as the difference between Pᵢ or pNP concentration in the extracts with and without incubation, expressed as nmol of Pᵢ or pNP released per min per mg protein. Phosphate was measured spectrophotometrically at 880 nm using the molybdenum-blue reaction at a fixed time (20 min) following addition of the color reagent (Carvalho et al., Ecotoxicol. Environ. Saf. 1998, 1:13-19). Phosphatase activity was determined from the release of pNP by measuring absorbance at 405 nm against standard solutions.

Arsenic and Phosphorus Analysis.

PV and PE tissues were dried at 60° C. for 96 h, weighed, and ground through a 2-mm mesh screen. Samples (0.1 g) were subjected to hot block digestion (USEPA Method 3051) and analyzed for total As using graphite furnace atomic absorption spectroscopy (GFAAS, Varian AA240Z, Walnut Creek, Calif.). Total P was calculated using the molybdenum blue method previously mentioned. To prevent interference of arsenate when using the molybdenum-blue method, samples were incubated with 300 μL 5% cysteine at 80° C. for 5 min to reduce arsenate to arsenite. Id.

Phytase Arsenic Resistance.

Arsenic tolerance of phytase and phosphatase enzymes was analyzed by performing previously described assays in the presence of arsenate. In addition to 5 mM P₆ or pNPP, plant extracts of PV, PE, and purified wheat phytase (WP) were incubated with 0, 0.5, 2, 2.5, and 5 mM arsenate.

Statistical Analysis.

Data are presented as the mean of all replicates and error bars represent one standard error either side of the mean. Significant differences were established by using One-way Analysis of Variance (ANOVA) and treatment means compared by Duncan's multiple range tests at p<0.05 (JMP® PRO, Version 10. SAS Institute Inc., Cary, N.C., 1989-2010).

### Results

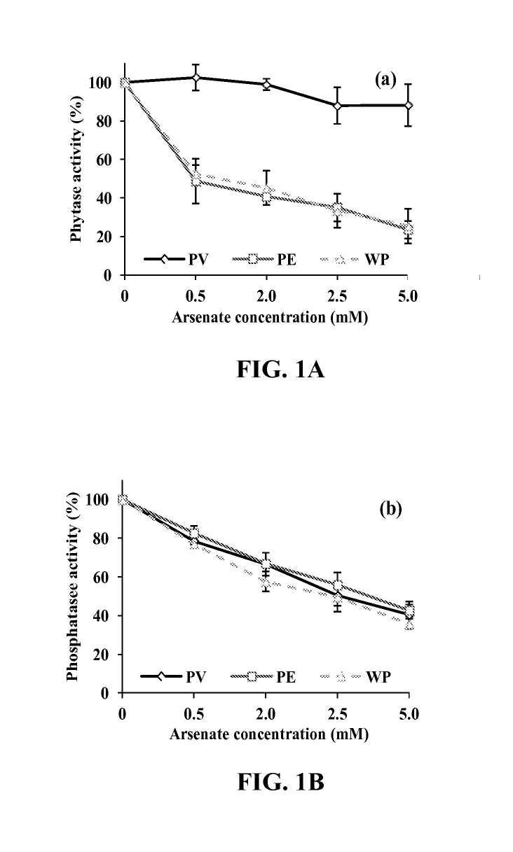

FIGS. 1A and 1B show the effect of arsenate on phytase (FIG. 1A) and phosphatase (FIG. 1B) activity in root extracts from PV, PE, and WP. Partial purification of PV phytase greatly increased its enzyme activities. The PV activities in the crude protein were 2.6 nmol Pᵢ and 8.6 nmol pNP mg⁻¹ protein min⁻¹. Ammonium sulfate precipitation followed by gel filtration increased activities by 9 to 26 fold. The highest purification was associated with the 20-40% ammonium sulfate fractions (68 nmol Pᵢ and 181 nmol pNP mg⁻¹ protein min⁻¹), which were used to estimate As tolerance.

Phytase and phosphatase activities were measured by production of Pᵢ and pNP hydrolyzed by the extracts of PV, PE and a crude wheat phytase in the presence of increasing concentrations of arsenate (0-5 mM). At 5 mM phytate or pNPP suspensions buffered at pH 5.0, enzyme activities for PV, PE, and WP were 46.7, 42.7, and 51.8 nmol Pᵢ mg⁻¹ protein min⁻¹ for phytase and 79.5, 149, and 163 nmol pNP mg⁻¹ protein min⁻¹ for phosphatase respectively (FIGS. 1A and 1B). Phytase activities in PV extracts were unaffected by arsenate up to 2 mM (46.7 to 46.1 nmol Pᵢ mg⁻¹ protein min⁻¹), with a slight decrease (˜41.1 nmol Pᵢ mg⁻¹ protein min⁻¹) at concentrations above 2.5 mM, which were not significantly different than the control (p<0.05; FIG. 1A). However, phytase activities from partially purified PE and wheat extracts exhibited a significant decrease with the addition of 0.5 mM arsenate, declining linearly (˜50, 43, 34 and 24% decrease) with increasing concentrations (FIG. 1A). Partial purification was done by ammonium sulfate precipitation followed by sephadex filtration. At 5 mM, their activities were ˜25% of the control. Phosphatase activities in extracts from PV, PE and crude wheat extracts were similarly impacted by arsenate, with significant declines at 0.5 mM As, decreasing to 36-45% of the control at 5 mM As (FIG. 1B).

Arsenate interferes with enzyme function, including phytases (Zhao et al., New Phytologist. 2008, 181:777-794). The shared homology of arsenate and phosphate allow for competitive inhibition, supported by the decline of PE and wheat phytase activities with increasing arsenate (FIG. 1A). However, PV phytase activity was unaffected by arsenate, which could be attributed to an alteration of the catalytic binding site. These results demonstrate that root-derived PV phytase may be resistant to other phytase inhibitors, such as other heavy metals.

### Example 3

### Thermostability of Root-Derived PV Phytase

### Materials and Methods

Enzyme collection, phytase assay, phosphotase assay, and statistical analysis were performed as in Example 2. Thermostability of enzyme activities was determined by pre-incubation of enzyme extracts in a water bath at 40, 60, 80, and 100° C. for 10 min.

### Results

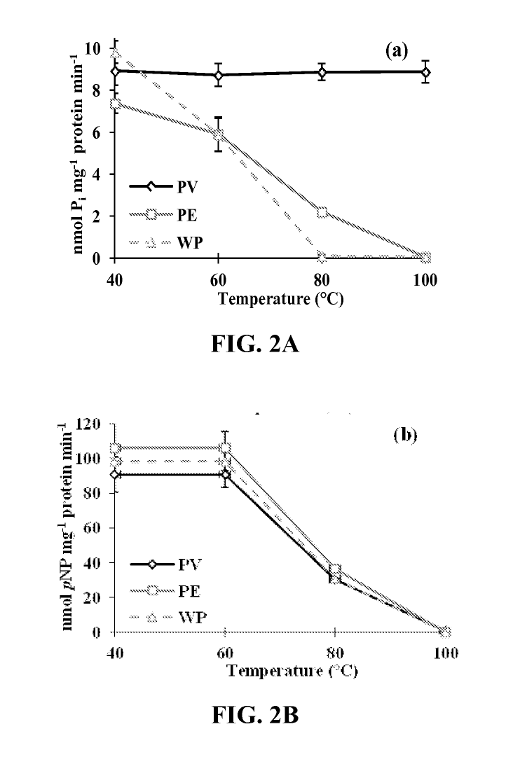

FIGS. 2A and 2B show the effect of temperature on root-derived PV phytase (FIG. 2A) or root-derived phosphatase (FIG. 2B). Partial purification of PV phytase greatly increased its enzyme activities. The PV activities in the crude protein were 2.6 nmol Pᵢ and 8.6 nmol pNP mg⁻¹ protein min⁻¹. Ammonium sulfate precipitation followed by gel filtration increased activities by 9 to 26 fold. The highest purification was associated with the 20-40% ammonium sulfate fractions (68 nmol Pᵢ and 181 nmol pNP mg⁻¹ protein min⁻¹), which were used to estimate thermostability. Phytase activities of PV extracts were unaffected (p<0.05) by all heat treatments compared to PE and WP, which lost significant activity at 60° C., decreasing to zero at 100° C. (FIG. 2A). Unexpectedly, the heat stability of PV phytase is unprecedented in plants. 100% of PV phytase activity was retained following 10 min pretreatments at 100° C. (FIG. 2A). Unlike phytase, phosphatase activities from enzyme extracts of all three plants decreased at a similar rate with increasing temperatures (FIG. 2B).

### Example 4

### Tissue Distribution of Phytase and Phosphatase in P. vittata and P. enisiformis

### Materials and Methods

Hydroponic Plant Culture. Two month old ferns, P. vittata (PV) and P. ensiformis (PE; a non As-hyperaccumulator), were placed in hydroponic culture in 0.2× strength Hoagland-Amon nutrient solution (HNS) for three weeks. Plants were rinsed with deionized (DI) water and transferred 500 mL 0.2× modified HNS with 0 or 210 μM Pᵢ (KH₂PO₄) and 0 or 267 μM As (Na₂HAsO₄.2H₂O) for 3 d. Treatments are referred to as control (No P), Pᵢ, As, and Pᵢ+As and were replicated four times.

Enzyme collection, phytase assay, phosphotase assay, and statistical analysis were performed as described in Example 2.

### Results

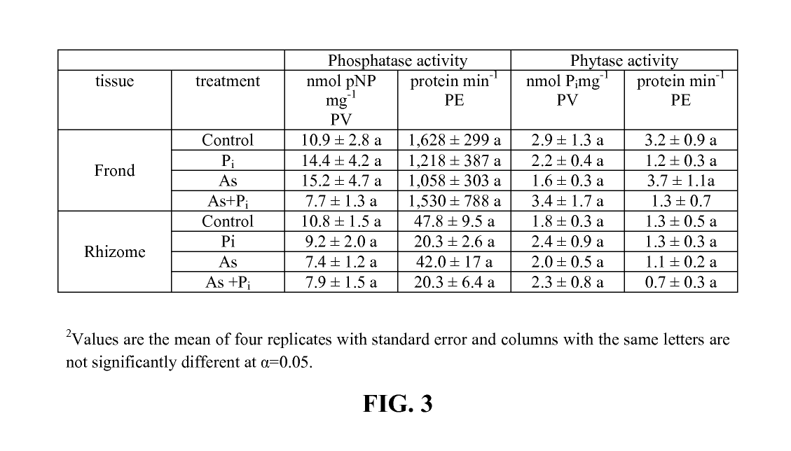

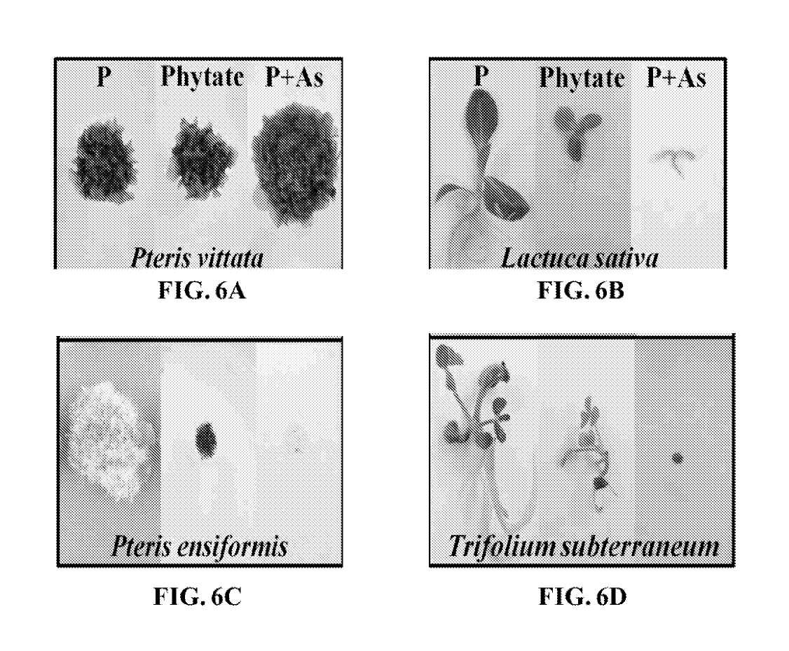

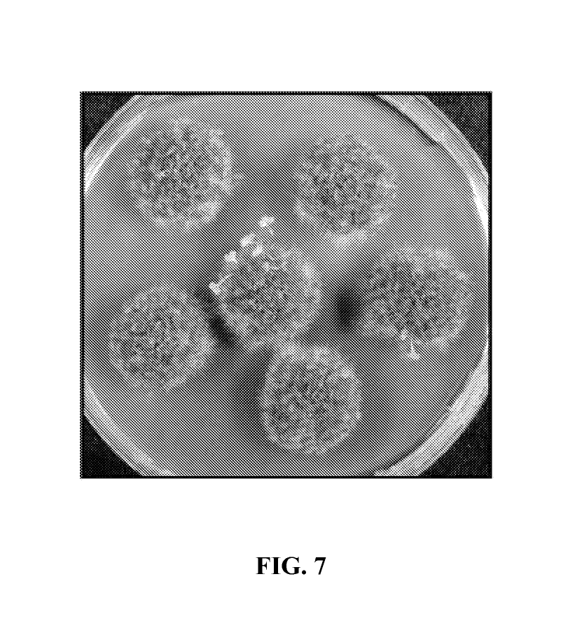

FIG. 6 shows the activities of phosphatase and phytase in frond and rhizome of PV and PE following 3-day treatment in phosphate and arsenate. FIG. 7 shows the activities of phosphatase and phytase in root tissue of PV and PE following 3-day treatment in phosphate and arsenate. After transplanting to media with Pᵢ, arsenate or both for 72 h, PV and PE showed no toxicity symptoms and phosphatase and phytase activities were detected in all tissues. Phosphatase activities in all treatments were much greater in PE than PV in all tissues, with the greatest difference in the fronds (85-198 times) (FIG. 3). Unlike phosphatase, phytase activities in all treatments were generally greater in PV, illustrating an inherent difference between the two species.

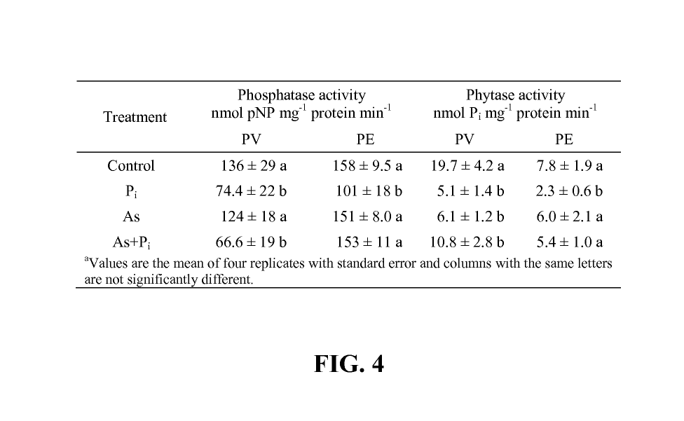

Neither Pᵢ or As treatment had significant impact on enzyme activities in the fronds or rhizomes of both PV and PE (FIG. 6). However, in the root extracts, enzyme activities responded to certain treatments. Removal of Pᵢ was the most effective treatment for increasing phosphatase and phytase activities in the roots. However, the addition of As nullified this response in PV root phytase activity, significantly reducing activity from 19.7 to 6.1 nmol Pᵢ mg⁻¹ protein min⁻¹ (FIG. 4).

During periods of P limitation, plants increase their internal phosphatase and phytase production to maintain Pᵢ levels (Sanchez-Calderon, L., et al. Phosphosrus: Plant Strategies to Cope with its Scarcity. In Cell Biology of Metals and Nutrients. 2010, p 173-198). When grown in the presence of As, plants often show symptoms of P-deficiency because arsenate competes with Pᵢ uptake and disrupts processes involving phosphorylation and phosphate signaling pathways (Abercrombie, J., et al. BCM Plant Biol. 2008, 8:87). This response was observed for phytase and phosphatase activity in PE root tissues, which were significantly elevated in the absence of Pᵢ or presence of As (p<0.05; FIG. 4).

Unexpectedly, this was not the case for PV root extracts, which did not respond to As-treatments. Since arsenate is a phosphate analog, PV roots may not differentiate between them. Instead, the metabolic and regulatory systems may have perceived the toxic metalloid as an abundant supply of Pᵢ, inhibiting the up-regulation of phytase production.

Enzyme activities of frond and rhizome tissues were unaffected by treatments, possibly because the 3 d incubation period was insufficient to elicit sufficient P-deficiency responses. Furthermore, P. ensiformis does not translocate As to the rhizome and frond. Alternatively, enzyme activity in both ferns may be associated with acquisition of Pᵢ from soil and not with internal P-homeostasis, which would explain why activity responses were not observed in frond and rhizome tissues (FIG. 3). Given the low activity of frond-derived phytase as compared with root-derived PV phytase, it is possible that these phytases are different from one another.

### Example 5

### Growth of P. vittata on Media Amended with Arsenic and Phytate

### Introduction

To estimate the ability of PV to utilize phytate as a sole source of Pᵢ its growth on modified MS media amended with either 0.6 mM Pᵢ and/or phytate (P₆) with and without 0.6 mM arsenate (Pᵢ+As, P₆+As, and Pᵢ+P₆+As) was compared to three angiosperms with known phytase activity (Lactuca sativa, Trifolium subterraneum, and Allium schoenoprasum) and two pteridophytes (P. ensiformis and T. kunthii).

### Materials and Methods

Seedling and Gametophyte Culture.

Seeds from Lactuca sativa, Trifolium subterraneum, and Allium schoenoprasum and spores from PE, Thelypteris kunthii, and PV were surface sterilized in a 20% bleach solution for 20 minutes followed by three washes in sterile DI water. Spores were suspended in 2 mL sterile DI water. Half strength modified Murashige & Skoog (MS) media was prepared with 0.8% agar without P prior to autoclaving. Phosphate, phytate, and arsenate solutions were filter sterilized and added to autoclaved MS media to obtain final concentrations of 0.6 mM P as Pᵢ or phytate (P₆; myo-inositol hexaphosphoric acid dodecasodium salt) with 0 or 0.6 mM arsenate. The MS media (pH 6.5) was then poured into sterile petri dishes (100 mm×13 mm). Seeds and spores (10 μL or 0.05 mg spore) were placed on agar (5 per plate, 4 plates per treatment) under cool/warm fluorescent lamps at 25° C. and 60% humidity for 15 and 40 d for seeded plants and ferns, respectively. Fresh weights were determined after growing plants for 15 d for angiosperms and 40 d for ferns.

Statistical analysis was performed as described in Example 2.

### Results

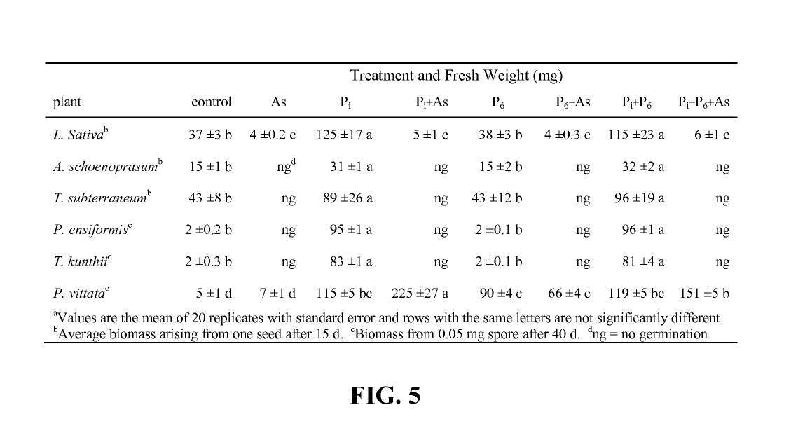

FIG. 5 shows the fresh weights of the plants (L. satvia, A. schoenoprasum, T. subterraneum, P. ensiformis, T. kunthii, and P. vittata) at 15 d or 40 d of growing. FIG. 6 shows plant growth at 15 d (L. sativa and T. subterraneum) or 40 d (PV and PE) in amended media. Germination rate for seeds were at least 90% and 100% for fern spores grown on modified MS media amended with 0.6 mM Pᵢ. Though all three angiosperms grew on P₆-amended media, their biomass production was significantly reduced (2.1-3.3 times) compared to the Pᵢ treatment (FIG. 5). The two comparative fern spores (PE and T. kunthii) germinated, but were unable to grow using P₆. However, growth of all plants on media amended with Pᵢ+P₆ were equivalent to Pᵢ treatments (p<0.05), verifying that the presence of P₆ had no negative effect on growth.

Pteris vittata was the only plant that effectively utilized P₆, maintaining biomass equivalent (p<0.05) to the Pᵢ treatment, and it survived beyond germination in the presence of 0.6 mM As (FIG. 5). Compared to the Pᵢ treatment, PV growth on Pᵢ+As was significantly increased (115 to 225 mg) while all treatments containing P₆ remained equivalent (p<0.05; FIG. 5).

Given the high enzymatic phytase activity in PV roots, especially under Pᵢ limiting conditions, we investigated whether PV spores could grow on sterile media amended with P₆ as the sole source of P. Phytate has been shown to be a poor source of P for plants due to both substrate availability and enzyme activity constraints (George, T., et al. Plant Cell Environ. M2004, 27:1351-1361 and Hayes, J., et al. Plant Soil. 2000, 165-174). This was not the case for PV, which grew equally well on Pᵢ or P₆ with equivalent total P after 40 d of growth (p<0.05). Furthermore, following 40 days of growth on all treatments, PV gametophytes produced sporophyte tissues (FIG. 7) showing that phytate utilization was not limited to the haploid growth stages. Most plants lack the ability to access external phytate because their phytases are confined to the endodermal region (Hayes, J., et al. Aust. J. Plant Physiol. 1999. 26:801-806) which was supported by the fact that other plants in our experiment produced comparable (p<0.05) biomass in phytate treatment as the control without P (FIG. 5). Even though T. subterraneum and L. sativa have been shown to increase root phytase activity in P-limiting and other stressful environments, (Id. and Nasri, N., et al. Acta Physiol. Plant. 2010, 33:935-942) they were unable to hydrolyze sufficient quantities of phytate in our experiment to sustain growth. The ability of PV to grow using phytate as a sole source of Pᵢ and the lack of growth in two other ferns suggests that phytate utilization is an adaptive trait specifically evolved in only some fern taxa.

In this example, PV was the only plant to survive beyond germination in the presence of 0.6 mM arsenate, which affected PV biomass and total P concentration depending on the source of P. After 40 d of growth, PV grown on Pᵢ+As agar were ˜2 times larger than all other treatments (p<0.05; FIG. 5). Despite having the largest biomass, PV tissue from Pᵢ+As agar had the lowest P concentration, which is consistent with previous findings that arsenate stimulates growth and competes with Pᵢ for uptake (Gumaelius, L., et al. Plant Physiol. 2004, 136:3198-3208).

### Example 6

### Increased Uptake of Phosphorus and Arsenic by P. vittata Gametophytes in Response to Low Available Phosphorus with Arsenic

### Materials and Methods

Seedling and Gametophyte culture was as described in Example 5. Statistical analysis was performed as described in Example 2.

### Results

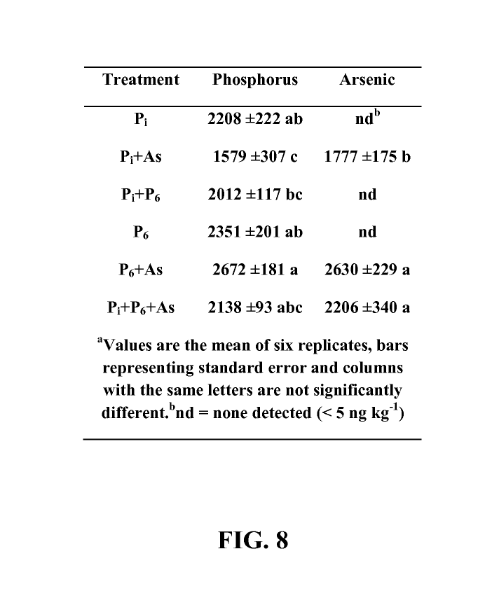

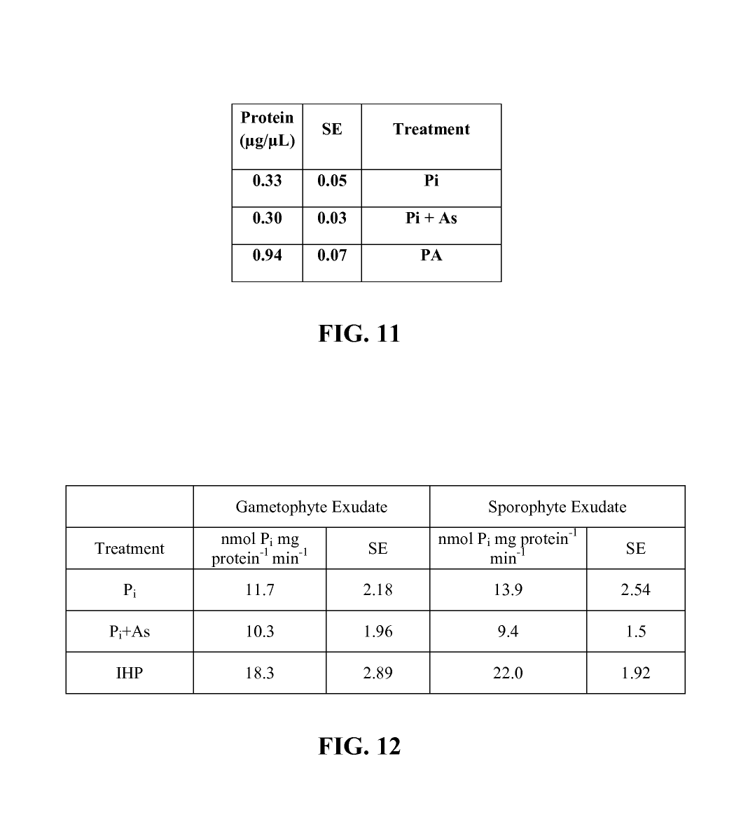

Total P and As in PV gametophytes grown on MS media with 0.6 mM Pᵢ, P₆ and/or As for 40 d are listed in FIG. 11. Average P concentrations in the Pᵢ treatment were 2,208 mg kg⁻¹ compared to 2,351 mg kg⁻¹ in the P₆ treatment, which were not significantly different indicating that PV gametophyte readily hydrolyzed and accumulated P from phytate. In the Pᵢ+As treatment, the total P and As tissue concentrations were 1,579 and 1,777 mg kg⁻¹ or 51 and 24 mmole kg⁻¹ respectively (FIG. 8). Compared to the Pᵢ+As treatment, concentrations of P and As in tissue from the P₆+As treatment were both significantly increased, which were 2,672 mg kg⁻¹ and 2,630 mg kg⁻¹ (p<0.05). This indicates that the low available P in P₆ coupled with As promoted up-regulation of P transporters, helping with both P and As uptake.

As compared to Example 5, arsenate had the opposite effect on gametophyte grown with phytate, reducing biomass below the Pᵢ control while significantly increasing total P (p<0.05; FIG. 8). Similar to the lack of phytase response in PV root tissue (FIG. 4), the presence of arsenate in the growth media may be perceived as Pᵢ (due to their homology) by PV gametophyte, delaying the transcriptional, physiological, and morphological responses that facilitate phytate hydrolysis.

Although growth was slowed, tissues from the P₆+As media had significantly higher concentrations of P and As compared to Pᵢ+As treatments (p<0.05; FIG. 8). Thus, grown on As-contaminated soils with Pₒ, maintaining low soluble Pᵢ could promote the uptake As, increasing the remediation capacity of PV. This also has the added benefit of removing the need for P fertilizers, for example, during phytoremediation. It should be noted that, after 80 d of growth, the initial slow growth of PV on P₆+As treatments abated, achieving weights equivalent to the Pᵢ treatments.

### Example 7

### Phytase Activity in P. vittata Root Exudates

Given that PV effectively utilized P₆ as a sole source of P for growth, phytase activity from gametophyte root exudates in response to P/As stress and P₆ was evaluated.

### Materials and Methods

Seedling and Gametophyte culture was as described in Example 5.

Tissue Collection and Enzyme Assays.

Root exudates were collected from the media of 40-day-old PV sporophytes. Root exudates were subjected to ammonium sulfate fractionation, collecting precipitates in 20% intervals from 0% to 80% fractions followed by gel filtration on Sephadex G-25, which was pre-equilibrated with 10 mM acetate buffer (pH 5.0). Phosphotase and Phytase activities were determined as described in Example 2.

Statistical analysis was performed as described in Example 2.

### Results

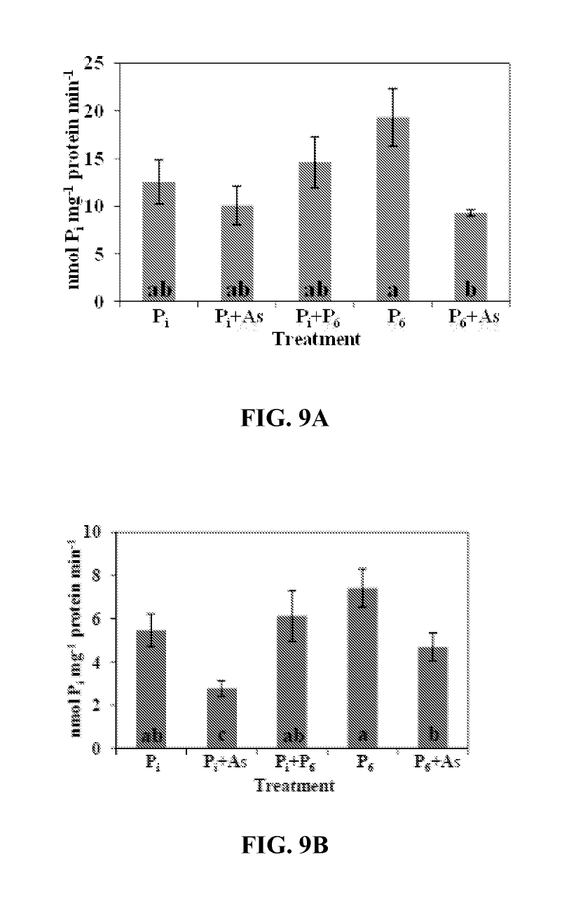

FIGS. 9A-9B show the effect of phytate on phytase activity in P. vittata root exudates (FIG. 9A) and gametophytes (FIG. 9B). Exudates collected from P₆ treatments exhibited the highest phytase activity. However, enzyme activities from all treatments were not significantly different from the Pᵢ control (FIGS. 9A and 9B). Compared to phytase activities in the root tissues (5.1 to 20 nmol Pᵢ mg⁻¹ protein min⁻¹; FIG. 7), those in the root exudates were comparable or higher (9.3 to 19 nmol Pᵢ mg⁻¹ protein min⁻¹), indicating that phytase in the root exudates likely accounted for the P acquisition. There was also an increase in the total protein content of exudates from P₆ and P₆+As (2.2 and 2.0 mg protein g⁻¹ tissue) compared to Pᵢ treatments (1.0, 1.1, and 1.0 mg protein g⁻¹ tissue for Pᵢ, Pᵢ+As and Pᵢ+P₆ respectively). However, since we used partially purified extracts, it is difficult to ascertain what percentage of the protein content is made up of phytase enzymes. Regardless, in the low P environment (P₆), there was an increase in protein exudation as shown in FIG. 11.

Phytase activity from tissue grown with phytate exhibited the highest phytase activities compared to Pᵢ+As treatments, which had the lowest (FIG. 9B). In gametophyte root exudates, phytase activities were ˜3 times greater than gametophyte tissues, again suggesting their role in P acquisition over internal remobilization. The phytase activity in gametophyte root exudates was comparable (p<0.05) to activity collected from sporophyte root exudates, indicating that P acquisition pathways exist in both reproductive stages of PV as shown in FIG. 15.

Associated activities were the highest in P₆ treatments, although not significantly different from the Pᵢ treatment. Thus, production and exudation of phytase enzymes in PV gametophyte appeared to be constitutive, regardless of Pᵢ availability. However, total protein content in exudates of P₆ and P₆+As treatments were double that of Pᵢ treatments, suggesting that PV responded to low available P environment by increasing total enzyme exudation.

### Example 8

### Root-Derived P. vittata Phytase Activity is not Deactivated by Soils

Most phytases in root exudate are deactivated by soils. As such, the effect of soils on activity of PV phytase was analyzed by measuring the rate of Pᵢ hydrolysis from phytate in solution following centrifugation. The amount of Pᵢ hydrolyzed represents phytase enzymes that are not sorbed to the soil matrix. For a comparative analysis, enzyme samples were incubated without soil (control) and soil samples were mixed with a non-enzymatic protein, bovine serum albumin (BSA), to estimate residual soil Pᵢ released from protein-soil interactions.

### Materials and Methods

Briefly, 2.0 g of air dried soil was mixed with DI water and enzyme extracts (or BSA as a negative control) to a 20 mL volume containing 50 μg protein per ml. Samples were placed on a rotary shaker (150 rpm) at room temperature. Enzyme abstracts were prepared from root tissue as described in Example 3. Aliquots of well-mixed soil slurry (250 μL) were removed using a pipette tip with a wide opening and centrifuged at 7,500 g for 5 min, using the supernatant (250 μL) to measure phytase activity after two hours. Phytase activity was determined as described in Example 2. Additional PV phytase activity measurements were taken after 6, 12, and 24 hours. Activities were derived from the difference between plant enzyme mediated Pᵢ release and the amount of Pᵢ in the BSA soil suspensions. Statistical analysis was performed as described in Example 2.

### Results

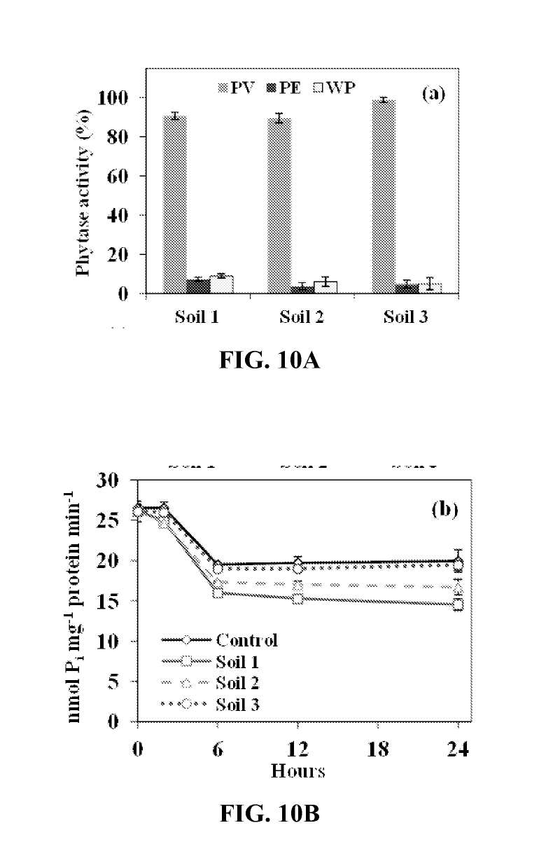

FIGS. 10A and 10B show phytase activity (FIG. 10A) remaining in soil suspensions after mixing with root-derived enzyme extracts from PV, PE, or WP for 2 h and the response of PV extracts to soil over a 24 h period (FIG. 10B). Root extracts from PV, PE and wheat phytase were mixed with three soils. Soil 1 was an acidic (pH 5.6) sand soil containing 2% OM with a cation exchange capacity (CEC) of 4.2 cmol⁺kg⁻¹. Soil 2 was an acidic (pH 5.8) loam soil with 0.8% OM and a CEC of 12.4 cmol⁺kg⁻¹. Soil 3 was a neutral (pH=6.5) clay soil with 0.4% OM and CEC of 24.8 cmol⁺kg⁻¹. Soil solutions mixed with BSA had no measurable Pᵢ throughout the experiment, indicating desorption of Pᵢ or native enzymes had no discernible impact on the results.

In the absence of soil, initial phytase activities averaged 26, 17 and 19 nmol Pᵢ mg⁻¹ protein min⁻¹ for PV, PE and wheat. After mixing with soils for 2 h, PV phytase was not significantly impacted, retaining 94, 93, and 98% of their activities in Soil 1, 2 and 3, respectively. In contrast, PE and wheat retained ˜6% activity in all soils after 2 h, which was not significantly different than zero (p<0.05; FIG. 7A). After 6 h, PV activity in the control declined 25%, where it was stable through 24 h, averaging ˜20 nmol Pᵢ mg⁻¹ protein min⁻¹ (FIG. 10B). A similar trend was observed with PV extracts mixed in the three soils, which retained 77, 86, and 97% of the control activity between 6 and 24 h for soil 1, 2 and 3 respectively. The addition of Soil 3 had no impact (p<0.05) on PV phytase activity compared to the control. Comparatively, there was a slight difference between the control and Soil 1 and 2, although it was <23%, representing decline between 6 and 24 h (p<0.05; FIG. 10B).

While plants have the capacity to exude phytases, sorption and precipitation reactions in soil limit their capacity to directly obtain Pᵢ from soil phytate (Brejnholt, S. M., et al. J. Sci. Food Agric. 2011, 91:1398-1405). This was not the case for PV enzymes, which retained 93-98% of their phytase activities after mixing with soils for 2 h compared to a >90% reduction in PE and wheat extracts, further illustrating the unique properties of PV phytases (FIGS. 10A and 10B). In similar soil studies, logarithmic decline of phytase activity is typically observed within minutes of soil addition (George, T. S., et al. Soil Biol. Biochem. 2005, 37:977-988). Following 24 h of mixing, PV extracts retained ˜64% of their original activity, which mirrored the decline in the soil-less control.

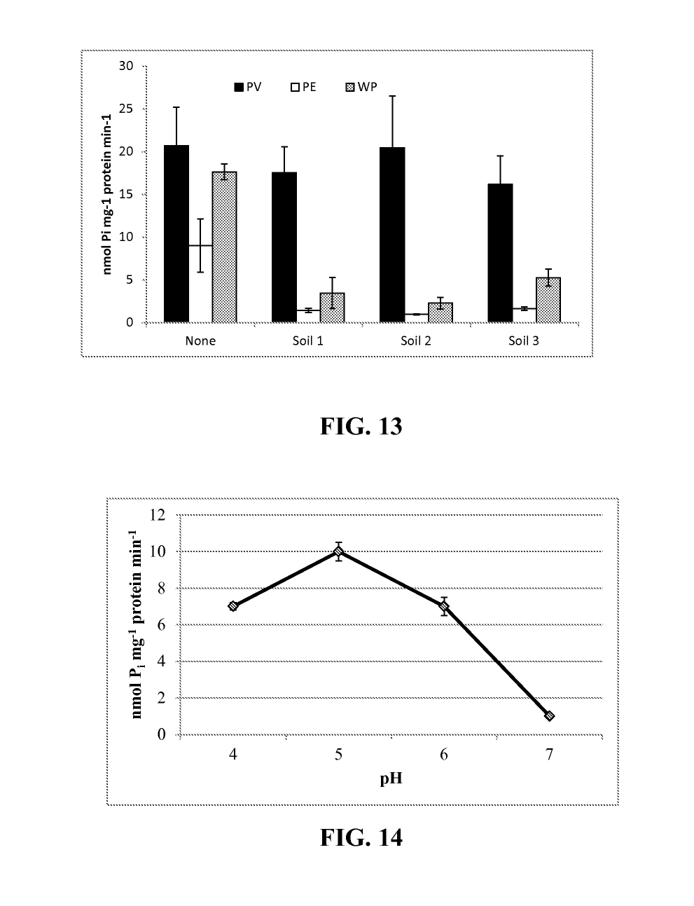

Normally, soils with greater clay content, organic matter and cation/anion exchange capacity more rapidly inhibit phytase activity (Rao, M. et al. Soil Biol. Biochem. 2000, 32:1007-1014). Even though Soil 3 had the greatest clay content and CEC, it did not effectively diminish PV phytase activity (FIG. 10B). Soil 1 and 2 had higher acidity and OM, which may have contributed to the slight decline in PV phytase activity. Under normal circumstances, sorption of phytase impairs the enzyme's ability to hydrolyze phosphate esters from phytate but root-derived PV phytases remained active even when sorbed to soil particles, as shown in FIG. 13, indicating a high affinity for phytate. This is significant because few plants can directly obtain Pᵢ from phytate in soils. The root-derived PV phytase thus does not have the limitations of other plant phytases, which are inactivated following exudation into the soil (Richardson, A. E., et al. Plant, Cell Environ. 2000, 23:397-405 and Hayes, J. E., et al. Aust. J. Plant Physiol. 1999, 26:801-809).

### Example 9

### Root-Derived PV Phytase has an Optimal pH of Approximately 5

### Materials and Methods

To assay optimal pH values for PV phytase activity, a series of 5 mM phytate solutions were prepared in different pH values. The assay buffers were prepared in 50 mM glycine-HCl (pH 3), acetic acid (pH 4 and 5), citrate (pH 6) and tris (pH 7). Incubation was carried out at 37° C. for 2 h.

### Results

As shown in FIG. 14, enzyme incubation at pH >7 resulted in loss of activity in PV extracts.

### Example 10

Pteris vittata ferns were grown hydroponically in nutrient solution amended with phytic acid as the sole source of P for 4 weeks. Actively growing root tips were excised and frozen in liquid nitrogen and stored at −80° C. RNA was extracted from root tissues using a Sigma Plant RNA extraction kit. RNA samples were sent to the Interdisciplinary Center for Biotechnology Research at the University of Florida. There, cDNA libraries were generated using Illumina MiSeq. Sequences with conserved purple acid phosphatase domains were identified and compared to known purple acid phosphatases using BLAST.

### DEFINITIONS

In describing the disclosed subject matter, the following terminology will is used in accordance with the definitions set forth below.

As used herein, “about,” “approximately,” and the like, when used in connection with a numerical variable, generally refers to the value of the variable and to all values of the variable that are within the experimental error (e.g., within the 95% confidence interval for the mean) or within. +−0.10% of the indicated value, whichever is greater.

As used herein, “nucleic acid” and “polynucleotide” generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotide” as that term is intended herein.

As used herein, “deoxyribonucleic acid (DNA)” and “ribonucleic acid (RNA)” generally refer to any polyribonucleotide or polydeoxribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes.

As used herein, “nucleic acid sequence” and “oligonucleotide” also encompasses a nucleic acid and polynucleotide as defined above.

As used herein, “deoxyribonucleic acid (DNA)” and “ribonucleic acid (RNA)” generally refer to any polyribonucleotide or polydeoxribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes.

As used herein, “gene” refers to a hereditary unit corresponding to a sequence of DNA that occupies a specific location on a chromosome and that contains the genetic instruction for a characteristic(s) or trait(s) in an organism.

As used herein, “locus” refers to the position that a given gene or portion thereof occupies on a chromosome of a given species.

As used herein, “allele(s)” indicates any of one or more alternative forms of a gene, where the alleles relate to at least one trait or characteristic. In a diploid cell or organism, the two alleles of a given gene occupy corresponding loci on a pair of homologous chromosomes.

The term “heterozygous” refers to a genetic condition where the organism or cell has different alleles at corresponding loci on homologous chromosomes.

As used herein, “homozygous” refers to a genetic condition where the organism or cell has identical alleles at corresponding loci on homologous chromosomes.

As used herein, the term “exogenous DNA” or “exogenous nucleic acid sequence” or “exogenous polynucleotide” refers to a nucleic acid sequence that was introduced into a cell, organism, or organelle via transfection. Exogenous nucleic acids originate from an external source, for instance, the exogenous nucleic acid may be from another cell or organism and/or it may be synthetic and/or recombinant. While an exogenous nucleic acid sometimes originates from a different organism or species, it may also originate from the same species (e.g., an extra copy or recombinant form of a nucleic acid that is introduced into a cell or organism in addition to or as a replacement for the naturally occurring nucleic acid). Typically, the introduced exogenous sequence is a recombinant sequence.

As used herein, the term “recombinant” generally refers to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a “fusion protein” (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc.). Recombinant also refers to the polypeptide encoded by the recombinant nucleic acid. Non-naturally occurring nucleic acids or polypeptides include nucleic acids and polypeptides modified by man.

As used herein, the term “transfection” refers to the introduction of an exogenous and/or recombinant nucleic acid sequence into the interior of a membrane enclosed space of a living cell, including introduction of the nucleic acid sequence into the cytosol of a cell as well as the interior space of a mitochondria, nucleus, or chloroplast. The nucleic acid may be in the form of naked DNA or RNA, it may be associated with various proteins or regulatory elements (e.g., a promoter and/or signal element), or the nucleic acid may be incorporated into a vector or a chromosome.

As used herein, “transformation” or “transformed” refers to the introduction of a nucleic acid (e.g., DNA or RNA) into cells in such a way as to allow expression of the coding portions of the introduced nucleic acid.

As used herein a “transformed cell” is a cell transfected with a nucleic acid sequence.

As used herein, a “transgene” refers to an artificial gene which is used to transform a cell of an organism, such as a bacterium or a plant.

As used herein, “transgenic” refers to a cell, tissue, or organism that contains a transgene.

As used herein, “polypeptides” or “proteins” are as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein “peptide” refers to chains of at least 2 amino acids that are short, relative to a protein or polypeptide.

As used herein, “variant” refers to a polypeptide that differs from a reference polypeptide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

As used herein, “functional variant” refers to a variant of a protein or polypeptide (e.g., a variant of a PGR5 protein) that can perform the same functions or activities as the original protein or polypeptide, although not necessarily at the same level (e.g., the variant may have enhanced, reduced or changed functionality, so long as it retains the basic function).

As used herein, “identity,” is a relationship between two or more polypeptide sequences, as determined by comparing the sequences. In the art, “identity” also refers to the degree of sequence relatedness between polypeptide as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453) algorithm (e.g., NBLAST, and XBLAST). The default parameters are used to determine the identity for the polypeptides of the present disclosure.

As used herein, “tolerant” or “tolerance” refers to the ability of a plant to overcome, completely or to some degree, the detrimental effect of an environmental stress or other limiting factor.

As used herein, “expression” as used herein describes the process undergone by a structural gene to produce a polypeptide. It is a combination of transcription and translation. Expression refers to the “expression” of a nucleic acid to produce a RNA molecule, but it is refers to “expression” of a polypeptide, indicating that the polypeptide is being produced via expression of the corresponding nucleic acid.

As used herein, “over-expression” and “up-regulation” refers to the expression of a nucleic acid encoding a polypeptide (e.g., a gene) in a transformed plant cell at higher levels (therefore producing an increased amount of the polypeptide encoded by the gene) than the “wild type” plant cell (e.g., a substantially equivalent cell that is not transfected with the gene) under substantially similar conditions.

As used herein, “under-expression” and “down-regulation” refers to expression of a polynucleotide (e.g., a gene) at lower levels (producing a decreased amount of the polypeptide encoded by the polynucleotide) than in a wild type plant cell.

As used herein, “inhibit” or “inhibiting” expression of a gene indicates that something (e.g., antisense nucleotide, suppressor, antagonist, etc.) acts to reduce or prevent (completely or partially) the transcription, translation and/or other processing step in the expression of a gene, thereby down-regulating the gene expression so that a reduced amount of the active protein encoded by the gene is produced as compared to wild type.

As used herein, “plasmid” as used herein refers to a non-chromosomal double-stranded DNA sequence including an intact “replicon” such that the plasmid is replicated in a host cell.

As used herein, the term “vector” or is used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both.

As used herein, “promoter” includes all sequences capable of driving transcription of a coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, “operatively linked” indicates that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and transcription control elements (e.g. promoters, enhancers, and termination elements), and/or selectable markers in an expression vector.

As used herein, “selectable marker” refers to a gene whose expression allows one to identify cells that have been transformed or transfected with a vector containing the marker gene. For instance, a recombinant nucleic acid may include a selectable marker operatively linked to a gene of interest and a promoter, such that expression of the selectable marker indicates the successful transformation of the cell with the gene of interest.

As used herein, “constitutive promoter” is a promoter that allows for continual or ubiquitous transcription of its associated gene or polynucleotide. Constitutive promoters are generally are unregulated by cell or tissue type, time, or environment.

As used herein, “inducible promoter” is a promoter that allows transcription of its associated gene or polynucleotide in response to a substance or compound (e.g. an antibiotic, or metal), an environmental condition (e.g. temperature), developmental stage, or tissue type.

As used herein, “wild-type” is the typical form of an organism, variety, strain, gene, protein, or characteristic as it occurs in nature, as distinguished from mutant forms that may result from selective breeding or transformation with a transgene.

As used herein, “electroporation” is a transformation method in which a high concentration of plasmid DNA (containing exogenous DNA) is added to a suspension of host cell protoplasts, and the mixture shocked with an electrical field of about 200 to 600 V/cm.

As used herein, “isolated” means separated from constituents, cellular and otherwise, in which the polynucleotide, peptide, polypeptide, protein, antibody, or fragments thereof, are normally associated with in nature. In one aspect of this disclosure, an isolated polynucleotide is separated from the 3′ and 5′ contiguous nucleotides with which it is normally associated with in its native or natural environment, e.g., on the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated,” “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the embodiments disclosed herein, it is to be understood that all of the above embodiments for each of the compositions disclosed below and under the appropriate conditions, are provided by this disclosure. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

As used herein, “cDNA” refers to a DNA sequence that is complementary to a RNA transcript in a cell. It is a man-made molecule. Typically, cDNA is made in vitro by an enzyme called reverse-transcriptase using RNA transcripts as templates.

As used herein, “purified” is used in reference to a nucleic acid sequence, peptide, or polypeptide that has increased purity relative to the natural environment.

As used herein, “control” is an alternative subject or sample used in an experiment for comparison purposes and included to minimize or distinguish the effect of variables other than an independent variable. A “control” can be positive or negative.

As used herein, “concentrated” used in reference to an amount of a molecule, compound, or composition, including, but not limited to, a chemical compound, polynucleotide, peptide, polypeptide, protein, antibody, or fragments thereof, that indicates that the sample is distinguishable from its naturally occurring counterpart in that the concentration or number of molecules per volume is greater than that of its naturally occurring counterpart.

As used herein, “diluted” used in reference to a an amount of a molecule, compound, or composition including but not limited to, a chemical compound, polynucleotide, peptide, polypeptide, protein, antibody, or fragments thereof, that indicates that the sample is distinguishable from its naturally occurring counterpart in that the concentration or number of molecules per volume is less than that of its naturally occurring counterpart.

As used herein, “separated” refers to the state of being physically divided from the original source or population such that the separated compound, agent, particle, chemical compound, or molecule can no longer be considered part of the original source or population.

As used herein, “synthetic” refers to a compound that is made by a chemical or biological synthesis process that occurs outside of and independent from the natural organism from which the compound can naturally be found.

1-65. (canceled)

66. A purified recombinant phytase having a polypeptide sequence having about 90% or greater sequence identity to any one of SEQ ID NOs: 3, 5, 7, 9, 11, 13, 15, 17, 19, or 21.

67. The purified recombinant phytase of claim 66, wherein the purified recombinant phytase catalyzes the release of phosphate from phytate.

68. The purified recombinant phytase of claim 66, wherein the purified recombinant phytase has about 50% or greater activity at a temperature greater than about 70 degrees Celsius.

69. The purified recombinant phytase of claim 66, wherein the purified recombinant phytase is contained in a cell.

70. The purified recombinant phytase of claim 66, wherein the cell is a is a plant, bacteria, yeast, or fungus cell.

71. The purified recombinant phytase of claim 66, wherein the purified recombinant phytase is contained in an animal feed.

72. The purified recombinant phytase of claim 66, wherein the purified recombinant phytase is contained in a fertilizer.

73. A cDNA molecule encoding a phytase from a root of Pteris vittata.

74. The cDNA molecule of claim 73 having 100% sequence identity to any one of SEQ ID NOs: SEQ ID NOs: 2, 8, 10, 12, 14, 16, 18, and 20.

75. The cDNA molecule of claim 73 having greater than or equal to about 90% sequence identity to any one of SEQ ID NOs: 2, 8, 10, 12, 14, 16, 18, and 20.

76. The cDNA molecule of claim 73, wherein the phytase has greater than or equal to about 50% activity at a temperature of greater than or equal to about 70 degrees Celsius.

77. The cDNA molecule of claim 73, wherein the cDNA molecule is operatively linked to a regulatory polynucleotide sequence.

78. The cDNA molecule of claim 73, wherein the cDNA molecule is contained in a vector.

79. The cDNA molecule of claim 73, wherein the cDNA molecule is contained in a cell.

80. The cDNA molecule of claim 73, wherein the cell is a is a plant, bacteria, yeast, or fungus cell.

81. A cDNA molecule encoding a polypeptide having greater than or equal to about 90% sequence identity to any one of SEQ ID NOs: 3, 9, 11, 13, 15, 17, 19, or 21.

82. The cDNA molecule of claim 81, wherein the cDNA molecule is operatively linked to a regulatory polynucleotide sequence.

83. The cDNA molecule of claim 81, wherein the cDNA molecule contained in a vector.

84. The cDNA molecule of claim 81, wherein the cDNA molecule is contained in a cell.

85. The cDNA molecule of claim 81, wherein the cell is a is a plant, bacteria, yeast, or fungus cell.


SEQUENCE LISTING (28 sequences)

SEQ ID NO: 1 - DNA, 1470 bp, Artificial Sequence
  root Pteris vittata phytase nucleotide sequence
tgcagctttcattacttgtaattatgcgcaagggcaataatataaatatgggcaaaggcattaataataatctagggctgcagctcggtctgatcatcctcattgcggctatggtctcccattacgcgaactcaggatatggcgaagcattcaggcaagcctcctcagctacggccaaaacaggctttgattccatcatttctcgccttggccatcaccaacctgacagtacaagcccgcaacaggtacatatctcattagcagggccacatcaaatcaaagtcacctggatctcatgtggttctgttcctatttctagggttgactatgggacaacacctggtttatatgacagatttgcagaagggagcagtgaatcttacacattcctattctataagtctggccaggttcacaatgttgttcttggccctttggaaccaaataccctttattactaccaatgcggaggcggaggtccagagtacagcttcaaaacaccaccacctatgggttcggaagttccagttacatttgcaatttctggtgatcttgggcagacagaatggacaacccaaacactaaatcgcattcaacaggaaaattatgatgtgctgatactgcctggagatctatcttatgctgattattatcagcctctctgggattcttttggacagcttgttgaacccttggcaagctcaagaccatggatggttacacaagggaaccatgaggtggaaagaatacctctgctaatagaacctttcagggcatacaacactcgctggcgaatgccttaccaagaaagtggttcagattcaaacctttattactcttttgaagttgctggtgcacatatccttatgcttggatcctatgcgaaattcgatccaggctcaaatcagtacaaatggcttctggctgatttggcgaatgtaaacagaaaagcaactccgtggctcatagcagtcatccatgcaccatggtatagcagcaattctgcgcatcagggtgatggtgaacaaatgaggcagtcaatggagcttattctgaaacaagcaaatgtcgatcttgtgtttgctggccatgtccatgcatatgagcggacggtgaaaatattcaactggaaggctgacgattgtggtgtttaccacattaccattggagatggtggaaaccgggagggcttagctcgcacttttctggaccctaagcctgattggtcggtttatcgagaggcaagttatggttatggtcttctagagatactgaactcaacccatgctcactggctttggcaccgcaatcaagacagcaatgcaattgtcggagacgatttgtggctattgaagaacccagaatcttcacaagtgtgtgtagcatcaggctagaacttgtctggtaaaaggtgtgtaaacatagctaggaaaggttagatgtgagacctaactgaagaanacatttgta

SEQ ID NO: 2 - DNA, 1347 bp, Artificial Sequence
  root pteris vittata phytase cDNA
atgggcaaaggcattaataataatctagggctgcagctcggtctgatcatcctcattgcggctatggtctcccattacgcgaactcaggatatggcgaagcattcaggcaagcctcctcagctacggccaaaacaggctttgattccatcatttctcgccttggccatcaccaacctgacagtacaagcccgcaacaggtacatatctcattagcagggccacatcaaatcaaagtcacctggatctcatgtggttctgttcctatttctagggttgactatgggacaacacctggtttatatgacagatttgcagaagggagcagtgaatcttacacattcctattctataagtctggccaggttcacaatgttgttcttggccctttggaaccaaataccctttattactaccaatgcggaggcggaggtccagagtacagcttcaaaacaccaccacctatgggttcggaagttccagttacatttgcaatttctggtgatcttgggcagacagaatggacaacccaaacactaaatcgcattcaacaggaaaattatgatgtgctgatactgcctggagatctatcttatgctgattattatcagcctctctgggattcttttggacagcttgttgaacccttggcaagctcaagaccatggatggttacacaagggaaccatgaggtggaaagaatacctctgctaatagaacctttcagggcatacaacactcgctggcgaatgccttaccaagaaagtggttcagattcaaacctttattactcttttgaagttgctggtgcacatatccttatgcttggatcctatgcgaaattcgatccaggctcaaatcagtacaaatggcttctggctgatttggcgaatgtaaacagaaaagcaactccgtggctcatagcagtcatccatgcaccatggtatagcagcaattctgcgcatcagggtgatggtgaacaaatgaggcagtcaatggagcttattctgaaacaagcaaatgtcgatcttgtgtttgctggccatgtccatgcatatgagcggacggtgaaaatattcaactggaaggctgacgattgtggtgtttaccacattaccattggagatggtggaaaccgggagggcttagctcgcacttttctggaccctaagcctgattggtcggtttatcgagaggcaagttatggttatggtcttctagagatactgaactcaacccatgctcactggctttggcaccgcaatcaagacagcaatgcaattgtcggagacgatttgtggctattgaagaacccagaatcttcacaagtgtgtgtagcatcaggctag

SEQ ID NO: 3 - protein, 456 aa, Artificial Sequence
  root pteris vittata phytase amino acid sequence
MRKGNNINMGKGINNNLGLQLGLIILIAAMVSHYANSGYGEAFRQASSATAKTGFDSIISRLGHHQPDSTSPQQVHISLAGPHQIKVTWISCGSVPISRVDYGTTPGLYDRFAEGSSESYTFLFYKSGQVHNVVLGPLEPNTLYYYQCGGGGPEYSFKTPPPMGSEVPVTFAISGDLGQTEWTTQTLNRIQQENYDVLILPGDLSYADYYQPLWDSFGQLVEPLASSRPWMVTQGNHEVERIPLLIEPFRAYNTRWRMPYQESGSDSNLYYSFEVAGAHILMLGSYAKFDPGSNQYKWLLADLANVNRKATPWLIAVIHAPWYSSNSAHQGDGEQMRQSMELILKQANVDLVFAGHVHAYERTVKIFNWKADDCGVYHITIGDGGNREGLARTFLDPKPDWSVYREASYGYGLLEILNSTHAHWLWHRNQDSNAIVGDDLWLLKNPESSQVCVASG

SEQ ID NO: 4 - DNA, 1410 bp, Artificial Sequence
  glycine max phytase cDNA (gi |358248461)
atgctgatgataatagattctcctgaacttgaagtacattgttgtcgtgctgctgatccaacgtttgcagaggtttctgttgtgttcacagttcacacacaaatggaactgaaactacttctaataacggttttaatgatggtgtcactttctgcaactgcagcagctgattacattcgacctcagcctcgaaaaaccttccatctcccatggcattctaaaccctcctcttaccctcaacaggtacacatttctttagcaggagaacagcacatgagagttacctggattactgatgataactctgcaccttcaattgtagaatatggaacatcaccagggcgatatgactctgtagctgaaggagaaaccacctcttacagttatctgttgtatagctcaggaaagatacaccatactgtaattgggcctttagagcataattctgtgtactattaccgatgtggtggacaaggtcctcagttccagctcagaactcctccagctcaacttccaatcacttttgccgtggctggtgatttgggtcaaaccggatggactaaatcaacattggatcacattgaccaatgtaaatataatgttcacctgcttccgggagacctttcatatgctgattatatccagcatcgctgggactcgtttggtaggctagtgcagccacttgctagtgctagaccatggatggtaacacaaggaaaccatgaagtagagagcatacctttgttaaaggatgggtttttgtcctataattccagatggaaaatgccatttgaggaaagcggatcaaattcaaatctctattattcgtttgaagttgcaggtgttcacattatcatgcttgggtcctatgcagattatgatgagtactctgaacaatatggatggctaaaggaagatctgtcaaaggtggatagggaaaggacaccttggttgattgtgttatttcatgtaccatggtataatagtaacacagctcatcaaggtgaaggggctgatatgatggcatctatggagccattgctttatgctgctagcgccgatttagttcttgccggtcatgtgcatgcttatgaacgctcaaaacgtgtatacaataaaagacttgatccttgtggttcagtccatataaccatcggtgatggaggaaacaaagaaggtttagctcctaagtatataaatccacagccaatatggtcagaattccgtgaagccagttttggtcatggtgagctacagattgtaaactctacgcatgctttctggagttggcacaggaatgatgatgatgagccagtaaaatccgatgatatctggataacatctttgaccagctcaggatgtgttgatcagaagagaaatgaactcaggaataaacttatgacaccttaa

SEQ ID NO: 5 - protein, 437 aa, Artificial Sequence
  glycine max phytase amino acid sequence (GI304421386)
MELKQQLLLLILTLLFATATPQYVRPLPRKTLTIPWDSISKAHSSYPQQVHISLAGDKHMRVTWITDDKHSPSYVEYGTLPGRYDSIAEGECTSYNYLLYSSGKIHHAVIGPLEDNTVYFYRCGGKGPEFELKTPPAQFPITFAVAGDLGQTGWTKSTLAHIDQCKYDVYLLPGDLSYADCMQHLWDNFGKLVEPLASTRPWMVTEGNHEEENILLLTDEFVSYNSRWKMPYEESGSTSNLYYSFEVAGVHVIMLGSYADYDVYSEQYRWLKEDLSKVDRKRTPWLLVLFHVPWYNSNKAHQGAGDDMMAAMEPLLYAASVDLVIAGHVHAYERSKRVYNGRLDPCGAVHITIGDGGNREGLAHKYINPQPKWSEFREASFGHGELKIVNSTHTFWSWHRNDDDEPVKADDIWITSLASSGCVDQKTHELRSTLLTP

SEQ ID NO: 6 - DNA, 1328 bp, Artificial Sequence
  Medicago truncatula phytase cDNA (gi|357459552)
atgaaactaaaactgattccaacagttttactgatcctgtcagtaacttcaactgctgatgactatgttagacctcagcctagaaaaaccttacatcttccatggcattctaaaccctcttcttatcctcaacaggtacacatttcattggcaggagacaagcatatgagggttacctggattactgacgacaaatctgcaccttcagttgttgaatacggaacattgccagggaagtatgacaatgtagctgaaggagagacaacctcttacagttacattttctacagctcaggaaagatacaccatacggtaataggtcctttagagccgaactctgtttacttttaccgatgcggtggactaggtccggagttcgagctcaaaacaccaccagctcaatttccaattagttttgctgtggttggagatcttggccaaactggttggaccaaatcaacgttagatcacatcgaccaatgcaagtacgatgttaacctgattcctggcgacctttcgtatgctgattatatccagcatcgttgggacacgtttggtagacttgtgcagccacttgctagttctagaccatggatggtaacacaagggaatcatgaagtagagcatatacctttgttaaaggatggatttatatcctataattcgagatggaaaatgccgtttgaggaaagtggatcaagctcgaacctctattattcttttgaagttgccggtgctcacattatcatgcttggttcatatgatgattatgatgtgtactcagaacaatataaatggctaaagacagatctgtcaaaggtggacaggaaaaggacaccttggttgcttgtcatatttcatgtcccatggtataatagtaacacagctcatcaaggtgaaggtggtgatatgatggaaaccatggagccattgctttatgctgctagcgtagatttagtttttgctggtcatgtccacgcatatgaacgctcgaaacgtgtatataatggaaaattggatccttgtggtgctgttcatataacaatcggtgatggagggaacaaagaaggcttagctcataagtatatagatccacagccgaagtggtcagagttccgtgaggccagtttcggtcatggtgagttaaagattgtaaattcaacgcatgccttctggagttggcaccggaatgacgatgacgagccggtaaaatctgatgatatctggataacctctttagttaactcaggatgtgttgctcagaagaaaactgaacttgagcatgcacttatgacaccctaaaatcccttgatcttctttatttcttc

SEQ ID NO: 7 - protein, 433 aa, Artificial Sequence
  Medicago truncatula Purple acid phosphatase amino acid sequence
MKLKLIPTVLLILSVTSTADDYVRPQPRKTLHLPWHSKPSSYPQQVHISLAGDKHMRVTWITDDKSAPSVVEYGTLPGKYDNVAEGETTSYSYIFYSSGKIHHTVIGPLEPNSVYFYRCGGLGPEFELKTPPAQFPISFAVVGDLGQTGWTKSTLDHIDQCKYDVNLIPGDLSYADYIQHRWDTFGRLVQPLASSRPWMVTQGNHEVEHIPLLKDGFISYNSRWKMPFEESGSSSNLYYSFEVAGAHIIMLGSYDDYDVYSEQYKWLKTDLSKVDRKRTPWLLVIFHVPWYNSNTAHQGEGGDMMETMEPLLYAASVDLVFAGHVHAYERSKRVYNGKLDPCGAVHITIGDGGNKEGLAHKYIDPQPKWSEFREASFGHGELKIVNSTHAFWSWHRNDDDEPVKSDDIWITSLVNSGCVAQKKTELEHALMTP

SEQ ID NO: 8 - DNA, 1356 bp, Artificial Sequence
  pteris vittata purple phosphatase 2 cDNA
atgggggacagcaagtgcatttatgtgttaatgctactactatgtatagtccattgtgacggtggagtaacaagctcctacaggcgcaagctcactgcgaccgtggatatgccgcttgacagtgatgtgtttaaggtccctgatgggtacaacgcaccagagcaggttcacatcacacaaggagaccttgagggaagagcagtcattgtatcgtggatcactccttctgaacctggatcgaatagggtttggtatagtttagaaaaaggagattatactcacagtgttgaaggaagggtgtcacagtacagatactacaactatacatccggatttatacaccgttgtaccataaagaacctccagcataacaagaagtacttttataagcttggggagggagattctgctcgggagttttggttttggactcccccaagtgtcaatcctgatgtggcttacacttttggaataatcggagatttggggcagacgtacgattccaacaaaaccttgcagcattatatgcaaggaaatgggcgggctcttctgtatgtaggggacctttcctatgccgataactatccaaatcatgataatagacgatgggatacttggggacgctttgtggagcctagcactgcgtaccagccttggatatggacagccggaaaccatgaactggactttgtacctgaactgggagagacggagccgttcaagccatatttaaaccgttaccatgttccatatcgtgcttccaacagcacatcacccctgtggtactccataaagagggcttcagcccacatcatcgtactttcatcatattcagcctatggaaaatatacaccacagtacagatggcttcagacagagttgacaaaggttaatagaaacaggaccccttggctgattgttttgttgcactctccatggtacaacagcaatggataccactacatggaaggcgagagtatgcgagttcagtttgaatcatggtttacggaggctaaggtcgatattgtgtttgcaggccatgtgcatgcatatgaacgatcgtatcgcgtttctaacatagcctacaacatagtgaatggggactgtttgcccaaactcaatgattcttcgcccgtttacgtgacaattggcgatggtggcaatgccgaaggtcttgccgcagagttcacagagccgcaaccagcttactctgcatttagagaggctagctttggccatgctatgcttgatatcaagaacaggacacatgccttctactattggcacaggaatgatgatgggaatgctgtggtggcagattcttattggctcaaaaaccaatattggcagagttga

SEQ ID NO: 9 - protein, 451 aa, Artificial Sequence
  pteris vittata purple acid phosphatase 2 amino acid sequence
MGDSKCIYVLMLLLCIVHCDGGVTSSYRRKLTATVDMPLDSDVFKVPDGYNAPEQVHITQGDLEGRAVIVSWITPSEPGSNRVWYSLEKGDYTHSVEGRVSQYRYYNYTSGFIHRCTIKNLQHNKKYFYKLGEGDSAREFWFWTPPSVNPDVAYTFGIIGDLGQTYDSNKTLQHYMQGNGRALLYVGDLSYADNYPNHDNRRWDTWGRFVEPSTAYQPWIWTAGNHELDFVPELGETEPFKPYLNRYHVPYRASNSTSPLWYSIKRASAHIIVLSSYSAYGKYTPQYRWLQTELTKVNRNRTPWLIVLLHSPWYNSNGYHYMEGESMRVQFESWFTEAKVDIVFAGHVHAYERSYRVSNIAYNIVNGDCLPKLNDSSPVYVTIGDGGNAEGLAAEFTEPQPAYSAFREASFGHAMLDIKNRTHAFYYWHRNDDGNAVVADSYWLKNQYWQS

SEQ ID NO: 10 - DNA, 1977 bp, Artificial Sequence
  pteris vittata purple acid phosphatase 3 cDNA
atggaactcgaaatgacaatgtggaacggggagggtccggaacgggagaagccaatgccaccaccccaaattattccgatacaatatcgcagacagcaaatttatggcatcatatggatgttgcttggactagccattgggattgggtcttgcaattcttatgtcgtgcatcgtcggaccgttttccatgaagaccaaccccttgcaaagattgctttacatcgcctgagttatgcgcaagattcatctgtgtccattgacgcatcaccaagtctacttggttccaagggtggatctgtggactatgtgaatgttcagttcaagaggccttcaggagcttctgcattggactggataggagttttctcaccggctgatttcaatgcttcactctgtacagcagatacagttggtacaaatcgggattatgcaccttttttgtgcactgcgcccatcaagtaccaatttgccaactttacaacccctaattatgtcgagacaggagaaggatcattgacatttcgtctcataaaccaacgtgcagatttctcctttgctttttttacaggaggtttggatgagcctgtactactggcagtcacaaatacaagtgtctcgtttgcaaatccaaaggctccttcatatccacgtcttgcgttgacgagcaaaacaaatgaggtgtctgtcacatggaccagtggttacagtcaaattgaggcggttccagtggtaaagtggggggagttaggtgccactgcggaaaccctcgtagtagctcaaactttgagttttgagcgctctgacatgtgtggttcacctgcaaggtcagtgggttggcgtgatccaggatacattcacactgcatttataggaggtctttggccgaatttcaagtacttctacaaggtaggacacaagctttcaaacgggacctttctatgggatcaagttaggaatttttcaggagctccatttccaggtgaagattctctgcagcaggttattatattcggtgacatgggaaagggagagcgagacctgtcaaacgaatataacaattttcagcctggagcactaaatacgacagactgcttgattgaagatcttaataacatagatctaatcttgcacataggagatctgtgctatgcgaacggttatctttctcagtgggaccaatttacagaacaaattgaaccattggcttcacatgtaccgtacatggtagcaagtggcaatcatgaaagagattggccgggcactggctcgttctatttaaacacagactctggtggggagtgtggtgtgctggcacagacaatgttcaacatgcctctgaaaaacagagaaaagttttggtactccatagattatggtctgtttcgtttttgcattgcggactccgaacatgactggcgagaaggcagcgagcaatataactttattgaggagtgctttgcatcagcggatcgcttgaaacaaccatggcttatctttacagcccatcgagtccttggttattcttcagataaatactatggtctggaaggcacatttggtgagcctatggcaagggaaagcctccaaaagctctggcagaaatataaggtggatttggcattctttggtcatgttcacaactatgagagaacttgccccgtttatgagagtacatgtgttagcacagaaacatctcattactcaggagtgttcaatgcaacaattcatctggtagtaggaggcgctggtgcaagcttatctgaatatagtgaagttcaaaccagctggagtgttttcaaggacctcgatcatggttttgggaaattgacttcattcaatcgatcagccttgctcttcgagtacaagcgcagcagcaatggtgaagtgtatgattctttttggatcaaaagggagtacaaagatgtactgggttgtgatattttaaataactgtccaccctttactctagctacgtaa

SEQ ID NO: 11 - protein, 658 aa, Artificial Sequence
  pteris vittata purple acid phosphatase 3 amino acid sequence
MELEMTMWNGEGPEREKPMPPPQIIPIQYRRQQIYGIIWMLLGLAIGIGSCNSYVVHRRTVFHEDQPLAKIALHRLSYAQDSSVSIDASPSLLGSKGGSVDYVNVQFKRPSGASALDWIGVFSPADFNASLCTADTVGTNRDYAPFLCTAPIKYQFANFTTPNYVETGEGSLTFRLINQRADFSFAFFTGGLDEPVLLAVTNTSVSFANPKAPSYPRLALTSKTNEVSVTWTSGYSQIEAVPVVKWGELGATAETLVVAQTLSFERSDMCGSPARSVGWRDPGYIHTAFIGGLWPNFKYFYKVGHKLSNGTFLWDQVRNFSGAPFPGEDSLQQVIIFGDMGKGERDLSNEYNNFQPGALNTTDCLIEDLNNIDLILHIGDLCYANGYLSQWDQFTEQIEPLASHVPYMVASGNHERDWPGTGSFYLNTDSGGECGVLAQTMFNMPLKNREKFWYSIDYGLFRFCIADSEHDWREGSEQYNFIEECFASADRLKQPWLIFTAHRVLGYSSDKYYGLEGTFGEPMARESLQKLWQKYKVDLAFFGHVHNYERTCPVYESTCVSTETSHYSGVFNATIHLVVGGAGASLSEYSEVQTSWSVFKDLDHGFGKLTSFNRSALLFEYKRSSNGEVYDSFWIKREYKDVLGCDILNNCPPFTLAT

SEQ ID NO: 12 - DNA, 1257 bp, Artificial Sequence
  pteris vittata purple acid phosphatase 4 cDNA
atgtcaatcatggcaatggcgatggtctggcactggttgtgggtgctgacccttctcctcggtggtagccaacgctccctccgatccgatctcttcttctcccgcagctccacgccctcccaggtgcatgtctcactagcaggccccaatggtatgagagtatcttgggttacaatcaatcatttatcaccatccatggtggagtttgggacaacatctggaatctatgacaaattagcctatggtgatagtgattcctacaattttttactttacagatctggccaaattcaccatgttgtactcaagaacctaagttccaacactctgtatttctacaaatgtggaggtgaaggcttggagtatacatttacaacaccaccggatgttggcccagaatcttcagttacatttgcggttgctggtgatctcggacaaactgacaatactcgttcaacactgaattacatcaagaagtcaaattatgatgtgcttctacttccgggggatctgtcttacgcggactattatcagccactgtgggacacctttggaactctgattgagccattagccagttcaaggccattaatggtgactcaaggaaaccacgagaaagagaatctccctcttgtcctagatcctttccgatcttacaatactcggtggagaatgccacatgaagagagtggttctgattcaaacctatattactcttttgaagtcacaggtgtccatgttctgatgcttggctcttatgcggacgttagtcgagactctagccagtacaaatggctgcaggatgatttggcaaaggtggaccgagacagaactccatggctcattgcagtccttcatgctccatggtacaacagcaataaaaaacatcagggtgatggtgatgagatgatgaggtccatggagtttcttttgtatgaagcaaaggttgatatactgtttgcgggtcatgtccatgcatatgaacgcacttcgagggttttcaatggcagaccggatttgtgtggcattacacacatcacagtaggagtcggcggcaacagagagggacttgctcatagctttttgagccctacgcctgagtggtccctttatcgagaagcgagttatggccatggtatcttgaagaaggtgaatgcaacccatgcatattggagctggcatcgaaatcaggatgatggcacagttatggcagatgagatatggctaaagagcaaggcaaatccatctacatgctga

SEQ ID NO: 13 - protein, 418 aa, Artificial Sequence
  pteris vittata purple acid phosphatase 4 amino acid sequence
MSIMAMAMVWHWLWVLTLLLGGSQRSLRSDLFFSRSSTPSQVHVSLAGPNGMRVSWVTINHLSPSMVEFGTTSGIYDKLAYGDSDSYNFLLYRSGQIHHVVLKNLSSNTLYFYKCGGEGLEYTFTTPPDVGPESSVTFAVAGDLGQTDNTRSTLNYIKKSNYDVLLLPGDLSYADYYQPLWDTFGTLIEPLASSRPLMVTQGNHEKENLPLVLDPFRSYNTRWRMPHEESGSDSNLYYSFEVTGVHVLMLGSYADVSRDSSQYKWLQDDLAKVDRDRTPWLIAVLHAPWYNSNKKHQGDGDEMMRSMEFLLYEAKVDILFAGHVHAYERTSRVFNGRPDLCGITHITVGVGGNREGLAHSFLSPTPEWSLYREASYGHGILKKVNATHAYWSWHRNQDDGTVMADEIWLKSKANPSTC

SEQ ID NO: 14 - DNA, 1500 bp, Artificial Sequence
  pteris vittata purple acid phosphatase 5 cDNA
atggtgaagcttcaacctttctgggtgttggtgctccttacttgctcaataggttgtgaaggtggtggagtgacaagctcgtatagacggaagctcgaggacaccaaagacatgccaatggacagtgatacttttaaagcaccagacggatataatgcgcctcaacaggtgcacataacacaaggagatgctgttggtacggccattattgtgacatggatcacaccctcagagcctggatccaacacggtttattatggtaaagagaatgggacatattccgattatgcagaagggaccttcaatcaatacaagttctacaattacacctcaggatttatacatcattgcactatcaagaaccttgagcacagcacgaagtatcattataaattaggtgaaggagactcagctcgagagttttggttttcgactcccccagaggtggatccagatatttcttacacattcggaattattggagatttgggccagacgtatgattcacaaaggactttccagcattatttgcagtcgaatggacaaacccttctttttgtgggggatctttcatatgctgataggtatcccttccatgacaataggcggtgggacacatggggccgtttcatcgagccaagtgctgcctaccaaccttggatatggacagcaggaaatcacgagcttgattttattccggatgtgggggaaagtgagccattcaaaccatacttgaacagatatcctacaccttatgattcctccaacagcacatcgccgttgtggtattccataaagagagcgtcagcacacattattgtgctttcgtcttattcagcatttggcacatactcgccccaatatcgatggctaaaggaagagctggctagtgtaaatcgaacaaagacaccatggctgatcatcctgatgcattctccgttctataacagcaacgagcatcactacatggagggtgaaaccatgagggttcaatttgaatcatggtttacagatgcaaaggttgacatcgtctttgctggtcatgtacatgcatatgagaggacatatcgaatttccaatgtggcctacaatattgttaatgggcaatgtactcctgtgcgcaatgagtctgcccctatgtatattacagttggtgatggtgggaactctgaaggccttgctgcagagttcacggagcctcagccaagctattctgccattagggaggctagctttgggcatggcatgcttgagataaaaaataggacacatgcttacttttattggcncaggatggtaacgctgtggtggccgactctgtatggcttggtaaccaatactggacttccaaggcacgaattaggacatacctttgaataccctcccatgaaaaatataaagaggaggaacaaggcaaggatagtgggtaaggtggtttgggttgccggagaagtgaattcgattcctaggggcattccagaaggacaaggagaaaactaa

SEQ ID NO: 15 - protein, 499 aa, Artificial Sequence
  pteris vittata purple acid phosphatase 5 amino acid sequence
MVKLQPFWVLVLLTCSIGCEGGGVTSSYRRKLEDTKDMPMDSDTFKAPDGYNAPQQVHITQGDAVGTAIIVTWITPSEPGSNTVYYGKENGTYSDYAEGTFNQYKFYNYTSGFIHHCTIKNLEHSTKYHYKLGEGDSAREFWFSTPPEVDPDISYTFGIIGDLGQTYDSQRTFQHYLQSNGQTLLFVGDLSYADRYPFHDNRRWDTWGRFIEPSAAYQPWIWTAGNHELDFIPDVGESEPFKPYLNRYPTPYDSSNSTSPLWYSIKRASAHIIVLSSYSAFGTYSPQYRWLKEELASVNRTKTPWLIILMHSPFYNSNEHHYMEGETMRVQFESWFTDAKVDIVFAGHVHAYERTYRISNVAYNIVNGQCTPVRNESAPMYITVGDGGNSEGLAAEFTEPQPSYSAIREASFGHGMLEIKNRTHAYFYWXRMVTLWWPTLYGLVTNTGLPRHELGHTFEYPPMKNIKRRNKARIVGKVVWVAGEVNSIPRGIPEGQGEN

SEQ ID NO: 16 - DNA, 1338 bp, Artificial Sequence
  pteris vittata purple acid phosphatase 6 cDNA
atgcccattgatagtgaggcattccgcgtccccaaggactcaaatgctccacaacaggtacatataacgcaaggagattatgatggccgtgcaatgattgtttcatgggtcaggacttcccaaagtgctgtacataatttatcatttgggctagcaaatggcgggtctttgtctagtaatgtgggctgcaacatcaccacttacacttttgcaaactacaagtctggatatattcatcattgtctcatacaaggccttgtgcatgatacgaattatacatacaagattgggcaaggaaacgaggctcgggaattctggttcatcacacctccagaacctggtccagatgcagcttacaattttggagtgattggagatttgggacaaacttatgattctcaaagaacatttgagcactataagacatcaagtgggcaaactgttctttttgttggtgatctttcttatgccgataattaccctcttgataatggagagcgctgggatacttggggacgctttgtcgagcctagtacagcttatcagccatggatttggacagcgggtaaccatgagattgaattcaggccagatcttggcgaagtaaaaccgttcaaacccttcttgcatcggtattccacaccttatttagcttccaagagcacatccccactttggtactccatacgaagagcctcggcacacattatcattctctcgagctattccgcttatggaaaatacacaccacaatgggtttggctacgtaatgagtttaaaagggtagaccgaaaagttacaccctggttaatagtcttgatgcatgcaccattgtacaatagcaatgtggctcattacatggagggggaaggcatgcgtgctgagttcgaggcttggttcttgaagtacagggtcgacattgtatttgcgggccacgttcatgcatatgaacgctctcatagaatagcaaacattgctagtgatatactaaatggaccacgggtacctgcacttgataagtcagcaccagtgtatgtaacaattggagatggtggcaacattgaggggcttgctagaatctacaaagatcctcaaccagattattcagcctttcgagaagccagctatggtcatgcaatgttagaaattaagaataggacacacgcacacctccattggcatcggaatgatgatggagaagcatcaattgccgatgaattctggctccagaatcagatctgggtaaaaagtgaattccaggatgtgaacccgaggcgaaagttgaagaagtcttttacacagctaaggtccaccatgtgtgagcagttaacagtttga

SEQ ID NO: 17 - protein, 445 aa, Artificial Sequence
  pteris vittata purple acid phosphatase 6 amino acid sequence
MPIDSEAFRVPKDSNAPQQVHITQGDYDGRAMIVSWVRTSQSAVHNLSFGLANGGSLSSNVGCNITTYTFANYKSGYIHHCLIQGLVHDTNYTYKIGQGNEAREFWFITPPEPGPDAAYNFGVIGDLGQTYDSQRTFEHYKTSSGQTVLFVGDLSYADNYPLDNGERWDTWGRFVEPSTAYQPWIWTAGNHEIEFRPDLGEVKPFKPFLHRYSTPYLASKSTSPLWYSIRRASAHIIILSSYSAYGKYTPQWVWLRNEFKRVDRKVTPWLIVLMHAPLYNSNVAHYMEGEGMRAEFEAWFLKYRVDIVFAGHVHAYERSHRIANIASDILNGPRVPALDKSAPVYVTIGDGGNIEGLARIYKDPQPDYSAFREASYGHAMLEIKNRTHAHLHWHRNDDGEASIADEFWLQNQIWVKSEFQDVNPRRKLKKSFTQLRSTMCEQLTV

SEQ ID NO: 18 - DNA, 1431 bp, Artificial Sequence
  pteris vittata purple acid phosphatase 7 cDNA
atggcgggatgctcagcaacggctgcctttccgctgtcgttcatactgatttggagctgctggcatttgggcacttccattgccactcccacttctccatgggcgctcaggggcagccataccagcctctatgtgcgctctcaggagtcctctgtggacatgcctcttgatgccgatgtctttgaagttccgccgggcttcaatgctcctcagcaggtacatattatgcaaggagactattatggcaaatcggttattgtctcgtggataacaccatgtgctgtggaatcggcaaatgtttactatggtacagataaggagaactattcttttacagcaacggcagagctgacaacaacatacacagcttacaactacacatctggttttatacatcattgcacactcgaggatctagagtataacacaacatatttctacaagcttggtgaggcaaatgtatctcgcgagttttcatttaccacgcctccagaatctggtcctgatgttccctacacatttggagttataggtgatcttgggcaaacagctgactccaatgccactttggagcattatgttcagagtagaggccagacccttctttttgtgggagactgttcttatgcagacaagtatcctttctatgataacaacagatgggatacctggggacgatttatcgaacgtagcgctgcttaccagccatggatctggactgtaggcaatcatgacattgaatttggtccatcctttggagagcttgatgagttcaaatcctacatccatcgactaattactccgtacaaagcatcacagagcacatcttccctttggtatgcaatccaacggggccctgcttacatcatttccctgtcatcgtactcctcttttgtgaaatacagtccacagtattattggctcaaatcagagttgaagaaagtggatcgaacaaagactccctggctgatcatcattatgcatgtgccgatttataacaccaactccaatcattaccttgaaggggaagctatgcggagtatctttgaagcctggtttgtggactacaaggttgacatcatttttgcaggtcatgttcatgcatatgagagaacgtatcctgtttccaatgttcgtttcaatttaacaaacgatgcctgccaaccgattttcaatgaagatgcgcctgtttatgttgtcattggggatggaggcaatgtagagggcttggccgtgccatacatagagccgcagccagcatactccgcatttcgagaagctagctttggccatggtttgcttgatataaagaattgcacgcatgccttattttcgtggcatcgcaaccaagatagggaggcagtggtggctgattctttttggctgaacaatcaatattggaagtcaagcagcagtttgcaagctacatga

SEQ ID NO: 19 - protein, 476 aa, Artificial Sequence
  pteris vittata purple acid phosphatase 7 amino acid sequence
MAGCSATAAFPLSFILIWSCWHLGTSIATPTSPWALRGSHTSLYVRSQESSVDMPLDADVFEVPPGFNAPQQVHIMQGDYYGKSVIVSWITPCAVESANVYYGTDKENYSFTATAELTTTYTAYNYTSGFIHHCTLEDLEYNTTYFYKLGEANVSREFSFTTPPESGPDVPYTFGVIGDLGQTADSNATLEHYVQSRGQTLLFVGDCSYADKYPFYDNNRWDTWGRFIERSAAYQPWIWTVGNHDIEFGPSFGELDEFKSYIHRLITPYKASQSTSSLWYAIQRGPAYIISLSSYSSFVKYSPQYYWLKSELKKVDRTKTPWLIIIMHVPIYNTNSNHYLEGEAMRSIFEAWFVDYKVDIIFAGHVHAYERTYPVSNVRFNLTNDACQPIFNEDAPVYVVIGDGGNVEGLAVPYIEPQPAYSAFREASFGHGLLDIKNCTHALFSWHRNQDREAVVADSFWLNNQYWKSSSSLQAT

SEQ ID NO: 20 - DNA, 1734 bp, Artificial Sequence
  pteris vittata purple acid phosphatase 8 cDNA
atggccgggagtgccaggcatcgtccatgtgtgcttctacggctcctccagcttcgaatttgcgcgctcctgctttctgtagcagcagtactgttgttgggtgctcccattggccatgcggacattccctccactgtggacggccccttcctgcccaagactgtggcgtacgatacatccctcgccaggggtagtgaggatctcccccattgggaccctcgccttgtcaagcgcgttccctccatttttcccgaacagatcgccctcgccctctccacccccgattccctgtgggtttcatggatcacaggagattctgaaattgggccaagcgtaatacccctagacccagcaagcgtggcgagcgaggtgcagtacggcacccaaagtggcaatcttacatgggtctccaccggaagtgcagaggtgtatagccagctttacccttacaagggtctgcttaattacacgtctggaatcattcatcatgttcgtatccaaggtctggctccgaacactaaatactattacagatgtggagatggtgctctgtctgctatgagtgaagagagatggttcagaactctgccacatcctagcccagataattacccaccaaagattgccatagtcggagatcttggactgacttataattcaagcagcaccttggatcacatagtgtggaatgagccctccttgcttcttatggttggagatctgagctatgccaatcagtatctcactataggcacaaagggagcgtcttgttacagttgcgagtttcctgattcacccacgcgggagacatatcagccgcattgggatgcatggggcaggttcatggagcctttgatttcgacagtgccaatgatggtaattgaggggaaccatgagattgaacctcaagctgacaacgtgacatttgcttcttacaaagcaagatttgccactccccatgaagaaagtggctcaggcactcaactgttttattcttacgaagcagggggcattcatttcattatgttgggaggctatgtggactacaaccgcacaggagaacaatttaggtggctacagaaagacctagagaaggtggatcgtgaggtaacaccttggttgatagccgcatggcaccccccctggtacaacagctacagatcacattatcgagaagtggagtgcatgaggctggagatggaaggtcttctttatgagcatggagtggatgcagttttctctggccacgttcacgcatatgagcgctctaatcgagtgtacaactacgaactgaatccttgtggtccagtatatataacagttggggatgggggcaacatagagatggtggatgtggtgcatgcggatgatgatgggcagtgcccgacaccattagattccatcccagagttcggaggaatatgccctttcaatttcaccgctggcccggcagcagggaagtattgctgggacacccagcctgaatggagtgctttcagggatggcagttttggccatggaatactggaggttctcaatgcgactcatgctctttggacatggcatcggaatcaagattttggaagaactgaacagcacggcgaccaaatctacattgttcgaacgcctgagctctgcccaaacttgcatacaaggaagcaagccatgtcaagagggacccatctatatgaaggagggcctgtatgcccaaagaaagttaatatgcagatttaa

SEQ ID NO: 21 - protein, 577 aa, Artificial Sequence
  pteris vittata purple acid phosphatase 8 amino acid sequence
MAGSARHRPCVLLRLLQLRICALLLSVAAVLLLGAPIGHADIPSTVDGPFLPKTVAYDTSLARGSEDLPHWDPRLVKRVPSIFPEQIALALSTPDSLWVSWITGDSEIGPSVIPLDPASVASEVQYGTQSGNLTWVSTGSAEVYSQLYPYKGLLNYTSGIIHHVRIQGLAPNTKYYYRCGDGALSAMSEERWFRTLPHPSPDNYPPKIAIVGDLGLTYNSSSTLDHIVWNEPSLLLMVGDLSYANQYLTIGTKGASCYSCEFPDSPTRETYQPHWDAWGRFMEPLISTVPMMVIEGNHEIEPQADNVTFASYKARFATPHEESGSGTQLFYSYEAGGIHFIMLGGYVDYNRTGEQFRWLQKDLEKVDREVTPWLIAAWHPPWYNSYRSHYREVECMRLEMEGLLYEHGVDAVFSGHVHAYERSNRVYNYELNPCGPVYITVGDGGNIEMVDVVHADDDGQCPTPLDSIPEFGGICPFNFTAGPAAGKYCWDTQPEWSAFRDGSFGHGILEVLNATHALWTWHRNQDFGRTEQHGDQIYIVRTPELCPNLHTRKQAMSRGTHLYEGGPVCPKKVNMQI

SEQ ID NO: 22 - DNA, 20 bp, Artificial Sequence
  forward primer for root pteris vittata phytase
ccttggcaagctcaagacca

SEQ ID NO: 23 - DNA, 20 bp, Artificial Sequence
  reverse primer for root pteris vittata phytase
atggacatggccagcaaaca

SEQ ID NO: 24 - DNA, 17 bp, Artificial Sequence
  degenerate forward primer for root PV cDNA amplification from PCR template
ggngayytnggncarac

SEQ ID NO: 25 - DNA, 20 bp, Artificial Sequence
  degenerate reverse primer for amplifying root PV cDNA from PCR template
tgccanswccartgngcrtg

SEQ ID NO: 26 - DNA, 20 bp, Artificial Sequence
  forward primer for RACE amplification of P. vittata phytase
ccttggcaagctcaagacca

SEQ ID NO: 27 - DNA, 20 bp, Artificial Sequence
  reverse primer for RACE amplification of P. vittata phytase
atggacatggccagcaaaca

SEQ ID NO: 28 - DNA, 20 bp, Artificial Sequence
  reverse primer for RACE amplification of P. vittata phytase
gccaaatcagccagaagcca